(19) United States
(12) Patent Application Publication
(10) Pub. No.: US 2006/0063818 A1
(43) Pub. Date: Mar. 23, 2006

(54) Hydantoin derivatives and deren verwendung als tace inhibitoren

(76) Inventors: Jeremy Nicholas Burrows, Macclesfield (GB); Howard Tucker, Macclesfield (GB)

Correspondence Address: FISH & RICHARDSON P.C., P.O BOX 1022, MINNEAPOLIS, MN, 55440-1022, US

(21) Appl. No.: 10/527,215

(22) PCT Filed: Sep. 9, 2003
(86) PCT No.: PCT/GB03/03914

§ 371 Date: Mar. 10, 2005

(30) Foreign Application Priority Data
Sep. 13, 2002 (GB) 0221246.2

(51) Int. Cl. A61K 31/427; A61K 31/422; A61K 31/4178; C07D 417/02; C07D 413/02; C07D 403/02

(52) U.S. Cl. 514/369; 514/376; 514/389; 548/183; 548/227; 548/314.7

(57) ABSTRACT

Hydantoin derivatives of Formula (1) that are useful in the inhibition of metalloproteinases and in particular in the inhibition of TNF-α Converting Enzyme (TACE).

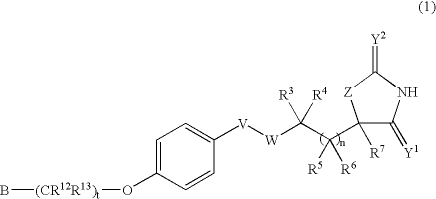

The present invention relates to compounds useful in the inhibition of metalloproteinases and in particular to pharmaceutical compositions comprising these, as well as their use.

The compounds of this invention are inhibitors of one or more metalloproteinase enzymes and are particularly effective as inhibitors of TNF-α (Tumour Necrosis Factor-α) production. Metalloproteinases are a superfamily of proteinases (enzymes) whose numbers in recent years have increased dramatically. Based on structural and functional considerations these enzymes have been classified into families and subfamilies as described in N. M. Hooper (1994) FEBS Letters 354:1-6. Examples of metalloproteinases include the matrix metalloproteinases (MMP) such as the collagenases (MMP1, MMP8, MMP13), the gelatinases (MMP2, MMP9), the stromelysins (MMP3, MMP10, MMP11), matrilysin (MMP7), metalloelastase (MMP12), enamelysin (MMP19), the Mr-MMPs (MMP14, MMP15, MMP16, MMP17); the reprolysin or adamalysin or MDC family which includes the secretases and sheddases such as TNF-α converting enzymes (ADAM10 and TACE); the ADAM-TS family (for example ADAM-TS1 and ADAM-TS4); the astacin family which include enzymes such as procollagen processing proteinase (PCP); and other metalloproteinases such as the endothelin converting enzyme family and the angiotensin converting enzyme family.

Metalloproteinases are believed to be important in a plethora of physiological disease processes that involve tissue remodelling such as embryonic development, bone formation and uterine remodelling during menstruation. This is based on the ability of the metalloproteinases to cleave a broad range of matrix substrates such as collagen, proteoglycan and fibronectin. Metalloproteinases are also believed to be important in the processing, or secretion, of biologically important cell mediators, such as tumour necrosis factor-α (TNF-α); and the post translational proteolysis processing, or shedding, of biologically important membrane proteins, such as the low affinity IgE receptor CD23 (for a more complete list see N. M. Hooper et al., (1997) Biochem J. 321:265-279).

Metalloproteinases have been associated with many disease conditions. Inhibition of the activity of one or more metalloproteinases may well be of benefit in these disease conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema and dermatitis); in tumour metastasis or invasion; in disease associated with uncontrolled degradation of the extracellular matrix such as osteoarthritis; in bone resorptive disease (such as osteoporosis and Paget's disease); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzheimer's disease; and extracellular matrix remodelling observed in cardiovascular diseases such as restenosis and atheroscelerosis.

A number of metalloproteinase inhibitors are known; different classes of compounds may have different degrees of potency and selectivity for inhibiting various metalloproteinases. We have discovered a class of compounds that are inhibitors of metalloproteinases and are of particular interest in inhibiting TACE. The compounds of this invention have beneficial potency and/or phannacokinetic properties.

TACE (also known as ADAM17) which has been isolated and cloned [R. A. Black et al. (1997) Nature 385:729-733; M. L. Moss et al. (1997) Nature 385:733-736] is a member of the admalysin family of metalloproteins. TACE has been shown to be responsible for the cleavage of pro-TNF-α, a 26 kDa membrane bound protein to release 17 kDa biologically active soluble TNF-α. [Schlondorff et al. (2000) Biochem. J. 347: 131-138]. TACE mRNA is found in most tissues, however TNF-α is produced primarily by activated monocytes, macrophages and T lymphocytes. TNF-α has been implicated in a wide range of pro-inflammatory biological processes including induction of adhesion molecules and chemokines to promote cell trafficking, induction of matrix destroying enzymes, activation of fibroblasts to produce prostaglandins and activation of the immune system [Aggarwal et al (1996) Eur. Cytokine Netw. 7: 93-124]. Clinical use of the anti-TNF-α biologicals has shown TNF-α to play an important role in a range of inflammatory diseases including rheumatoid arthritis, Crohn's disease and psoriasis [Onrust et al (1998) Biodrugs 10: 397422, Jarvis et al (1999) Drugs 57:945-964]. TACE activity has also been implicated in the shedding of other lscbrarie bound proteins including TGFα, p 75 & p 55 TNF receptors, L-selectin and amyloid precursor protein [Black (2002) Int. J. Biochem. Cell Biol. 34: 1-5]. The biology of TACE inhibition has recently been reviewed and shows TACE to have a central role in TNF-α production and selective TACE inhibitors to have equal, and possibly greater, efficacy in the collagen induced arthritis model of RA than strategies that directly neutralise TNF-α [Newton et al (2001) Ann. Rheum. Dis. 60: iii25-iii32].

A TACE inhibitor might therefore be expected to show efficacy in all disease where TNF-α has been implicated including, but not limited to, inflammatory diseases including rheumatoid arthritis and psoriasis, autoimmune diseases, allergic/atopic diseases, transplant rejection and graft versus host disease, cardiovascular disease, reperfusion injury, malignancy and other proliferative diseases. A TACE inhibitor might also be useful in the treatment of respiratory disorders such as asthma and chronic obstructive pulmonary diseases (referred to herein as COPD).

TACE inhibitors are known in the art. WO 02/096426 describes hydantoin derivatives which are useful as inhibitors of matrix metalloproteinases, TACE, aggrecanase, or a combination thereof.

We are able to provide further compounds that have metalloproteinase inhibitory activity, and are in particular inhibitors of TACE (ADAM17).

The present invention provides a compound of formula (1), a pharmaceutically acceptable salt or in vivo hydrolysable ester thereof: 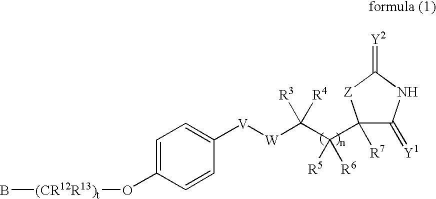
wherein:
Y¹ and Y² are independently O or S;
z is NR⁸, O or S;
n is 0 or 1;
W is NR¹, CR¹R²or a bond;
V is C(═O), NR¹⁵C(═O), NR¹⁵SO₂, SO₂ or a group of formula (A): 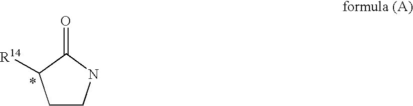
where the group of formula (A) is bonded through nitrogen to W of formula (1) and through carbon * to phenyl of formula (1);
t is 0 or 1;
B is a group selected from aryl, heteroaryl and heterocyclyl where each group is optionally substituted by one or more groups independently selected from nitro, trifluoromethyl, trifluoromethoxy, halo, cyano, C₁₋₄alkyl (optionally substituted by R⁹ or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl, heterocyclyl whereby this group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl and C₁₋₄alkoxy; with the provisos that:
when V is a group of formula (A), C(═O), NR¹⁵C(═O) or NR¹⁵SO₂; or when V is SO₂ and n is 1 and W is NR¹, CR¹R² or a bond; or when V is SO₂ and n is 0 and W is CR¹R²; then B is a group selected from aryl, heteroaryl and heterocyclyl where each group is optionally substituted by one or more groups independently selected from nitro, trifluoromethyl, trifluoromethoxy, halo, cyano, C₁₋₄alkyl (optionally substituted by R⁹ or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl, heterocyclyl whereby this group is optionally substituted by one or more halo, nitro, cyano, trifluoromethyl, trifluoromethoxy, —CONOR⁹, —CONR⁹R¹⁰, —SO₂R¹¹, —SO₂NR⁹R¹⁰, —NR⁹SO₂R¹¹, C₁₋₄alkyl and C₁₋₄alkoxy; and
when V is SO₂ and n is 0 and W is NR¹ or a bond; then B is a group selected from bicyclic aryl , bicyclic heteroaryl and bicyclic heterocyclyl, where each group is optionally substituted by one or more groups independently selected from nitro, trifluoromethyl, trifluoromethoxy, halo, cyano, C₁₋₄alkyl (optionally substituted by R⁹ or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —GONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl, heterocyclyl whereby this group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl and C₁₋₄alkoxy;
R¹ and R² are independently hydrogen or a group selected from C₁₋₆alkyl, C₂₋₆alkenyl, C₂₋₆alkynyl, C₃₋₆cycloalkyl and C₅₋₆cycloalkenyl where the group may be optionally substituted by halo, cyano, nitro, hydroxy or C₁₋₄alkoxy;
R³, R⁴, R⁵ and R⁶ are independently hydrogen or a group selected from C₁₋₆alkyl, C₂₋₆alkenyl, C₂₋₆amlyl, C₃₋₆cycloalkyl, C₅₋₆cycloalkenyl, aryl, heteroaryl and heterocyclyl where the group is optionally substituted by one or more substituents independently selected from halo, nitro, cyano, trifluoromethyl, trifluoromethyloxy, C₁₋₄alkyl, C₂₋₄alkenyl, C₂₋₄alkynyl, C₃₋₆cycloalkyl (optionally substituted by one or more R¹⁷), aryl (optionally substituted by one or more R¹⁷), heteroaryl (optionally substituted by one or more R¹⁷), heterocyclyl, —OR¹⁸, —SR¹⁹, —SOR¹⁹, —SO₂R¹⁹, —COR¹⁹, —CO₂R¹⁸, —CONR¹⁸R²⁰, —NR¹⁶COR¹⁸, —SO₂NR¹⁸R²⁰ and —NR¹⁶SO₂R¹⁹;
or R¹ and R³ together with the nitrogen or carbon and carbon to which they are respectively attached form a saturated 3- to 7-membered ring optionally containing 1 or 2 heteroatoms groups selected from NH, O, S, SO and SO₂ where the ring is optionally substituted on carbon or nitrogen by one or more C₁₋₄alkyl;
or R³ and R⁴ together form a saturated 3- to 7-membered ring optionally containing a heteroatom group selected from NH, O, S, SO and SO₂ where the ring is optionally substituted on carbon or nitrogen by one or more C₁₋₄alkyl;
or R³ and R⁵ together with the carbon atoms to which they are attached form a saturated 3- to 7-membered ring optionally containing a heteroatom group selected from NH, O, S, SO and SO₂ where the ring is optionally substituted on carbon or nitrogen by one or more C₁₋₄alkyl;
or R⁵ and R⁶ together form a saturated 3- to 7-membered ring optionally containing a heteroatom group selected from NH, O, S, SO and SO₂ where the ring is optionally substituted on carbon or nitrogen by one or more C₁₋₄alkyl;
R⁷ is hydrogen or a group selected from C₁₋₆alkyl, C₂₋₆alkenyl, C₂₋₆alkynyl, heteroalkyl, C₃₋₇cycloalkyl, aryl, heteroaryl or heterocyclyl where the group is optionally substituted by halo, C₁₋₄alkyl, C₁₋₄alkoxy, C₃₋₇cycloalkyl, heterocyclyl, aryl, heteroaryl and heteroalkyl; and
wherein the group from which R⁷ may be selected is optionally substituted on the group and/or on its optional substituent by one or more substituents independently selected from halo, cyano, C₁₋₄alkyl, nitro, haloC₁₋₄alkyl, heteroalkyl, aryl, heteroaryl, hydroxyC₁₋₄alkyl, C₃₋₇cycloalkyl, heterocyclyl, C₁₋₄alkoxyC₁₋₄alkyl, haloC₁₋₄alkoxyC₁₋₄alkyl, carboxyC₁₋₄alkyl, —OR²¹, —CO₂R²¹, —SR²⁵, —SOR²⁵, —SO₂R²⁵, —NR²¹COR²², —CONR²¹R²² and —NHCONR²¹R²²;
or R³ and R⁷ together with the carbon atoms to which they are each attached and (CR⁵R⁶)ₙ form a saturated 5- to 7-membered ring optionally containing a heteroatom group selected from NH, O, S, SO and SO₂ where the ring is optionally substituted on carbon or nitrogen by one or more C₁₋₄alkyl;
R⁸ is selected from hydrogen, C₁₋₆alkyl and haloC₁₋₆alkyl;
R⁹ and R¹⁰ are independently hydrogen, C₁₋₆alkyl or C₃₋₆cycloalkyl;
or R⁹ and R¹⁰ together with the nitrogen to which they are attached form a heterocyclic 4- to 7-membered ring;
R¹¹ is C₁₋₆alkyl or C₃₋₆cycloalkyl;
R¹² and R¹³ are independently selected from hydrogen, C₁₋₆alkyl and C₃₋₆cycloalkyl;
R¹⁴ is hydrogen, —NR²³R²⁴ or C₁₋₄alkyl (optionally substituted by halo, —OR²³ and —NR²³R²⁴);
R¹⁶, R²³ and R²⁴ are independently hydrogen or C₁₋₆alkyl;
R¹⁷ is selected from halo, C₁₋₆alkyl, C₃₋₆cycloalkyl and C₁₋₆alkoxy;
R¹⁸ is hydrogen or a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl where the group is optionally substituted by one or more halo;
R¹⁹ and R²⁵ are independently a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl where the group is optionally substituted by one or more halo;
R²⁰ is hydrogen, C₁₋₆alkyl or C₃₋₆cycloalkyl;
or R¹⁸ and R²⁰ together with the nitrogen to which they are attached form a heterocyclic 4- to 7-membered ring;
R²¹ and R²² are independently hydrogen, C₁₋₄alkyl, haloC₁₋₄alkyl, aryl, arylC₁₋₄alkyl and benzoyl.

In particular, the present invention provides a compound of formula (1) or a pharmaceutically acceptable salt thereof wherein: 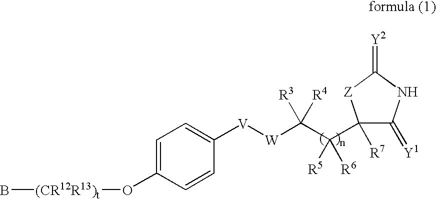
Y¹ and Y² are both O;
z is NR⁸, O or S;
n is 0 or 1;
W is CR¹R² or a bond;
V is a group of formula (A): 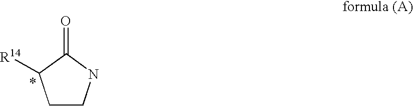
where the group of formula (A) is bonded through nitrogen to W of formula (1) and through carbon * to phenyl of formula (1);
t is 0 or 1;
B is a group selected from aryl, heteroaryl and heterocyclyl where each group is optionally substituted by one or more groups independently selected from nitro, trifluoromethyl, trifluoromethoxy, halo, cyano, C₁₋₄alkyl (optionally substituted by R⁹ or C₁₋₄alkoxy or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl and heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl and C₁₋₄alkoxy;
R¹ and R² are independently hydrogen or a group selected from C₁₋₆alkyl, C₂₋₆alkenyl, C₂₋₆alkynyl, C₃₋₆cycloalkyl and C₅₋₆cycloalkenyl which group may be optionally substituted by halo, cyano, hydroxy or Ci₄alkoxy;
R³, R⁴, R⁵ and R⁶ are independently hydrogen or a group selected from C₁₋₆alkyl, C₂₋₆alkenyl, C₂₋₆alkmnyl, C₃₋₆cycloalkyl, C₅₋₆cycloalkenyl, aryl, heteroaryl and heterocyclyl which group is optionally substituted by one or more substituents independently selected from halo, nitro, cyano, trifluoromethyl, trifluoromethoxy, C₁₋₄alkyl, C₂₋₄alkenyl, C₂₋₄alkynyl, C₃₋₆cycloalkyl (optionally substituted by one or more R¹⁷), aryl (optionally substituted by one or more R¹⁷), heteroaryl (optionally substituted by one or more R¹⁷), heterocyclyl, —OR¹⁸, —SR¹⁹, —SOR¹⁹, —SO₂R¹⁹, COR¹⁹, —CO₂R¹⁸, —CONR¹⁸R²⁰, —NR¹⁶CR¹⁸, —SO₂NR¹⁸R²⁰ and —NR¹⁶SO₂R¹⁹;
or R¹ and R³ together with the carbon atoms to which they are attached form a saturated 3- to 7-membered ring optionally containing 1 or 2 heteroatoms groups selected from NH, O, S, SO and SO₂ where the ring is optionally substituted on carbon by C₁₋₄alkyl, fluoro or C₁₋₃alkoxy and/or on nitrogen by —COC₁₋₃alkyl or —SO₂C₁₋₃aLkyl or one or more C₁₋₄alkyl;
or R³ and R⁴ together with the carbon atom to which they are attached form a saturated 3- to 7-membered ring optionally containing a heteroatom group selected from NH, O, S, SO and SO₂ where the ring is optionally substituted on carbon by C₁₋₄alkyl, fluoro or C₁₋₃alkoxy and/or on nitrogen by —COC₁₋₃alkyl or —SO₂C₁₋₃alkyl or C₁₋₄alkyl;
or R³ and R⁵ together with the carbon atoms to which they are attached form a saturated 3- to 7-membered ring optionally containing a heteroatom group selected from NH, O, S, SO and SO₂ where the ring is optionally substituted on carbon by C₁₋₄alkyl, fluoro or C₁₋₃alkoxy and/or on nitrogen by —COC₁₋₃alkyl or —SO₂C₁₋₃alkyl or C₁₋₄alkyl;
or R⁵ and R⁶ together with the carbon atom to which they are attached form a saturated 3- to 7-membered ring optionally containing a heteroatom group selected from NH, O, S, SO and SO₂ where the ring is optionally substituted on carbon by C₄₋₁alkyl, fluoro or C₁₋₃alkoxy and/or on nitrogen by —COC₁₋₃alkyl or —SO₂C₁₋₃alkyl or C₁₋₄alkyl;
R⁷ is hydrogen or a group selected from C₁₋₆alkyl, C₂₋₆alkenyl, C₂₋₆alkynyl, heteroalkyl, C₃₋₇cycloalkyl, aryl, heteroaryl or heterocyclyl which group is optionally substituted by halo, C₁₋₄alkyl, C₁₋₄alkoxy, C₃₋₇cycloalkyl, heterocyclyl, aryl, heteroaryl and heteroalkyl; and wherein the group from which R⁷ may be selected is optionally substituted on the group and/or on its optional substituent by one or more substituents independently selected from halo, cyano, C₁₋₄alkyl, nitro, haloC₁₋₄alkyl, heteroalkyl, aryl, heteroaryl, hydroxyC₁₋₄alkyl, C₃₋₇cycloalkyl, heterocyclyl, C₁₋₄alkoxyC₁₋₄alkyl, haloC₁₋₄alkoxyC₁₋₄alkyl, —COC₁₋₄alkyl, —OR²¹, —R²¹R²², —CO₂R²¹, —SR²⁵, —SOR²⁵, —SO₂R²⁵, —NR²¹COR²², —CONR²¹R²² and —NHCONR²¹R²²;
or R³ and R⁷ together with the carbon atoms to which they are each attached and (CR⁵R⁶)ₙ form a saturated 5- to 7-membered ring optionally containing a heteroatom group selected from NH, O, S, SO and SO₂ where the ring is optionally substituted on carbon by C₁₋₄alkyl, fluoro or C₁₋₃alkoxy and/or on nitrogen by —COC₁₋₃alkyl or —SO₂C₁₋₃alkyl or C₁₋₄alkyl;
R⁸ is hydrogen or methyl;
R⁹ and R¹⁰ are independently hydrogen, C₁₋₆alkyl or C₃₋₆cycloalkyl;
or R⁹ and R¹⁰ together with the nitrogen to which they are attached form a heterocyclic 4- to 7-membered ring;
R¹¹ is C₁₋₆alkyl or C₃₋₆cycloalkyl;
R¹² and R¹³ are independently selected from hydrogen, C₁₋₆alkyl and C₃₋₆cycloalkyl;
R¹⁴ is hydrogen, nitrile, —NR²³R²⁴ or C₁₋₄alkyl (optionally substituted by halo, —OR²³ and —NR²³R²⁴);
R¹⁶, R²³ and R²⁴ are independently hydrogen or C₁₋₆alkyl;
R¹⁷ is selected from halo, C₁₋₆alkyl, C₃₋₆cycloalkyl and C₁₋₆alkoxy;
R¹⁸ is hydrogen or a group selected from C₁₋₆alkyl, C₃₋₆cycloallyl, C₅₋₆cycloalkenyl, saturated heterocyclyl, aryl, heteroaryl, arylC₁₋₄alkyl and heteroarylC₁₋₄alkyl which group is optionally substituted by one or more halo;
R¹⁹ and R²⁵ are independently a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl which group is optionally substituted by one or more halo;
R²⁰ is hydrogen, C₁₋₆alkyl or C₃₋₆cycloalkyl;
or R¹⁸ and R²⁰ together with the nitrogen to which they are attached form a heterocyclic 4- to 7-membered ring;
R²¹ and R²² are independently hydrogen, C₁₋₄alkyl, haloC₁₋₄alkyl, aryl and arylC₁₋₄alkyl.

As a further aspect an in vivo hydrolysable ester of a compound of formula (1) is provided.

It is to be understood that, insofar as certain of the compounds of formula (1) defined above may exist in optically active or racemic forms by virtue of one or more asymmetric carbon or sulphur atoms, the invention includes in its definition any such optically active or racemic form which possesses metalloproteinases inhibition activity and in particular TACE inhibition activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

Compounds of formula (1) are therefore provided as enantiomers, diastereomers, geometric isomers and atropisomers.

Within the present invention it is to be understood that a compound of formula (1) or a salt thereof may exhibit the phenomenon of tautomerism and that the formulae drawings within this specification can represent only one of the possible tautomeric forms. It is to be understood that the invention encompasses any tautomeric form which has metalloproteinases inhibition activity and in particular TACE inhibition activity and is not to be limited merely to any one tautomeric form utilised within the formulae drawings.

It is also to be understood that certain compounds of formula (1) and salts thereof can exist in solvated as well as unsolvated forms such as, for example, hydrated forms. It is to be understood that the invention encompasses all such solvated forms which have metalloproteinases inhibition activity and in particular TACE inhibition activity.

It is also to be understood that certain compounds of formula (1) may exhibit polymorphism, and that the invention encompasses all such forms which possess rnetalloproteinases inhibition activity and in particular TACE inhibition activity.

The present invention relates to compounds of formula (1) as defined herein as well as to the salts thereof. Salts for use in pharmaceutical compositions will be pharmaceutically acceptable salts, but other salts may be useful in the production of compounds of formula (1) and their pharmaceutically acceptable salts. Pharmaceutically acceptable salts of the invention may, for example, include acid addition salts of compounds of formula (1) as defined herein which are sufficiently basic to form such salts. Such acid addition salts include but are not limited to hydrochloride, hydrobromide, citrate and maleate salts and salts formed with phosphoric and sulphuric acid. In addition where compounds of formula (1) are sufficiently acidic, salts are base salts and examples include but are not limited to, an alkali metal salt for example sodium or potassium, an alkaline earth metal salt for example calcium or magnesium, or organic amine salts for example triethylamine or tris-(2-hydroxyethyl)amine.

The compounds of formula (1) may also be provided as in vivo hydrolysable esters. An in vivo hydrolysable ester of a compound of formula (1) containing a carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

Suitable pharmaceutically acceptable esters for hydroxy include inorganic esters such as phosphate esters (including phosphoramidic cyclic esters) and a-acyloxyalkyl ethers and related compounds which as a result of the in vivo hydrolysis of the ester breakdown to give the parent hydroxy group/s. Examples of a-acyloxyalkyl ethers include acetoxymethoxy and 2,2-dimethylpropionyloxymethoxy. A selection of in vivo hydrolysable ester forming groups for hydroxy include C₁₋₁₀alkanoyl, for example formyl, acetyl; benzoyl; phenylacetyl; substituted benzoyl and phenylacetyl, C₁₋₁₀alkoxycarbonyl (to give alkyl carbonate esters), for example ethoxycarbonyl; di-(C₁₋₄)alkylcarbamoyl and N-(di-(C₁₋₄)alkylaminoethyl)-N-(C₁₋₄)alkylcarbamoyl (to give carbamates); di-(C₁₋₄)alkylaminoacetyl and carboxyacetyl. Examples of ring substituents on phenylacetyl and benzoyl include aminomethyl, (C₁₋₄)alkylaminomethyl and di-((C₁₋₄)alkyl)aminomethyl, and morpholino or piperazino linked from a ring nitrogen atom via a methylene linking group to the 3- or 4-position of the benzoyl ring. Other interesting in vivo hydrolysable esters include, for example, R^{A}C(O)O(C₁₋₆)alkyl-CO—, wherein R^{A} is for example, benzyloxy-(C₁₋₄)alkyl, or phenyl). Suitable substituents on a phenyl group in such esters include, for example, 4-(C₁₋₄)piperazinyl-(C₁₋₄)alkyl, piperazinyl-(C₁₋₄)alkyl and morpholino-(C₁₋₄)alkyl.

In this specification the generic term “alkyl” includes both straight-chain and branched-chain alkyl groups. However references to individual alkyl groups such as “propyl” are specific for the straight chain version only and references to individual branched-chain alkyl groups such as ter-butyl are specific for the branched chain version only. For example, “C₁₋₃alkyl” includes methyl, ethyl, propyl and isopropyl, examples of “C₁₋₄alkyl” include the examples of “C₁₋₃alkyl” and butyl and tert-butyl and examples of “C₁₋₆alkyl” include the examples of “C₁₋₄alkyl” and additionally pentyl, 2,3-dimethylpropyl, 3-methylbutyl and hexyl. An analogous convention applies to other genetic terms, for example “C₂₋₄alkenyl” includes vinyl, allyl and 1-propenyl and examples of “C₂₋₆alkenyl” include the examples of “C₂₋₄alkenyl” and additionally 1-butenyl, 2-butenyl, 3-butenyl, 2-methylbut-2-enyl, 3-methylbut-1-enyl, 1-pentenyl, 3-pentenyl and 4-hexenyl. Examples of “C₂₋₄alkynyl” includes ethynyl, 1-propynyl, 2-propynyl, 3-butynyl and examples of “C₂₋₆alkynyl” include the examples of “C₂₋₄alkynyl” and additionally 2-pentynyl, hexynyl and 1-methylpent-2-ynyl. Where examples are given for generic terms, it should be noted that these examples are not limiting.

“Cycloalkyl” is a monocyclic, saturated alkyl ring. The term “C₃₋₄cycloalkyl” includes cyclopropyl and cyclobutyl. The term “C₃₋₅cycloalkyl” includes “C₃₋₄cycloalkyl and cyclopentyl. The term “C₃₋₆cycloalkyl” includes “C₃₋₅cycloalkyl”, and cyclohexyl. The term “C₃₋₇cycloalkyl” includes “C₃₋₆cycloalkyl” and additionally cycloheptyl. The term “C₃₋₁₀cycloalkyl” includes “C₃₋₇cycloalkyl” and additionally cyclooctyl, cyclononyl and cyclodecyl.

“Cycloalkenyl” is a monocyclic ring containing 1, 2, 3 or 4 double bonds. Examples of “C₅₋₆cycloalkenyl” are cyclopentenyl, cyclohexenyl and cyclohexadiene and examples of “C₅₋₁₀cycloalkenyl” include the examples of “C₅₋₆cycloalkenyl” and cyclooctatriene.

Unless otherwise specified “aryl” is monocyclic or bicyclic. Examples of “aryl” therefore include phenyl (an example of monocyclic aryl) and naphthyl (an example of bicyclic aryl).

Examples of “arylC₁₋₄alkyl” are benzyl, phenylethyl, naphthylmethyl and naphthylethyl.

Unless otherwise specified “heteroaryl” is a monocyclic or bicyclic aryl ring containing 5 to 10 ring atoms of which 1, 2, 3 or 4 ring atoms are chosen from nitrogen, sulphur or oxygen where a ring nitrogen or sulphur may be oxidised. Examples of heteroaryl are pyridyl, imidazolyl, quinolinyl, cinnolyl, pyrimidinyl, thienyl, pyrrolyl, pyrazolyl, thiazolyl, oxazolyl, isoxazolyl, pyrazinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl and pyrazolopyridinyl. Preferably heteroaryl is pyridyl, imidazolyl, quinolinyl, pyrimidinyl, thienyl, pyrazolyl, thiazolyl, oxazolyl and isoxazolyl. More preferably heteroaryl is pyridyl, imidazolyl and pyrimidinyl. Examples of “monocyclic heteroaryl” are pyridyl, imidazolyl, pyrimidinyl, thienyl, pyrrolyl, pyrazolyl, thiazolyl, oxazolyl, isoxazolyl and pyrazinyl. Examples of “bicyclic heteroaryl” are quinolinyl, quinazolinyl, cinnolinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl and pyrazolopyridinyl. Preferred examples B when B is heteroaryl are those examples of bicyclic heteroaryl.

Examples of “heteroarylC₁₋₄alkyl” are pyridylmethyl, pyridylethyl, pyrimidinylethyl, pyrimidinylpropyl, pyrimidinylbutyl, imidazolylpropyl, imidazolylbutyl, quinolinylpropyl, 1,3,4-triazolylpropyl and oxazolylmethyl.

“Heterocyclyl” is a saturated, unsaturated or partially saturated, monocyclic or bicyclic ring (unless otherwise stated) containing 4 to 12 atoms of which 1, 2, 3 or 4 ring atoms are chosen from nitrogen, sulphur or oxygen, which may, unless otherwise specified, be carbon or nitrogen linked, wherein a —CH₂— group can optionally be replaced by a —C(O)—; and where unless stated to the contrary a ring nitrogen or sulphur atom is optionally oxidised to form the N-oxide or S-oxide(s); a ring —NH is optionally substituted by acetyl, formyl, methyl or mesyl; and a ring is optionally substituted by one or more halo. Examples and suitable values of the term “heterocyclyl” are piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, pyranyl, dihydro-2H-pyranyl, tetrahydrofuranyl, 2,5-dioximidazolidinyl, 2,2-dimethyl-1,3-dioxolanyl and 3,4-dimethylenedioxyphenyl. Preferred values are 3,4-dihydro-2H-pyran-5-yl, tetrahydrofuran-2-yl, 2,5-dioximidazolidinyl, 2,2-dimethyl-1,3-dioxolan-2-yl and 3,4-methylenedioxyphenyl. Other values are pyridoimnidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinoline, tetrahydroisoquinoline and isoindolinyl. Examples of monocyclic heterocyclyl are piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, pyranyl, tetrahydrofuranyl, 2,5-dioximidazolidinyl and 2,2-dimethyl-1,3-dioxolanyl. Examples of bicyclic heterocyclyl are pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl, isoindolinyl. 2,3-methylenedioxyphenyl, and 3,4-methylenedioxyphenyl. Examples of saturated heterocyclyl are piperidinyl, pyrrolidinyl and morpholinyl.

The term “halo” refers to fluoro, chloro, bromo and iodo.

Examples of “C₁₋₃alkoxy” and “C₁₋₄alkoxy” include methoxy, ethoxy, propoxy and isopropoxy. Examples of “C₁₋₆alkoxy” include the examples of “C₁₋₄alkoxy” and additionally pentyloxy, 1-ethylpropoxy and hexyloxy.

“Heteroalkyl” is alkyl containing at least one carbon atom and having at least one carbon atom replaced by a hetero group independently selected from N, O, S, SO, SO₂, (a hetero group being a hetero atom or group of atoms). Examples include —CH₂OCH₃, —CH₂SH and —OC₂H₅.

“HaloC₁₋₄alkyl” is a C₁₋₄alkyl group substituted by one or more halo. Examples of “haloC₁₋₄alkyl” include fluoromethyl, trifluoromethyl, 1-chloroethyl, 2-chloroethyl, 2-bromopropyl, 1-fluoroisopropyl and 4-chlorobutyl. Examples of “haloC₁₋₆alkyl” include the examples of “haloC₁₋₄aLkyl” and 1-chloropentyl, 3-chloropentyl and 2-fluorohexyl.

Examples of “hydroxyC₁₋₄alkyl” include hydroxymethyl, 1-hydroxyethyl, 2-hydroxyethyl, 2-hydroxypropyl, 1-hydroxyisopropyl and 4-hydroxybutyl.

Example of “C₁₋₄alkoxyC₁₋₄akyl” include methoxymethyl, ethoxymethyl, methoxyethyl, methoxypropyl and propoxybutyl.

“HaloC₁₋₄alkoxyC₁₋₄alkyl” is a C₁₋₄alkoxyC₁₋₄alkyl group substituted on C₁₋₄alkoxy by one or more halo. Examples of “haloC₁₋₄alkoxyC₁₋₄alkyl” include 1-(chloromethoxy)ethyl, 2-fluoroethoxymethyl, trifluoromethoxymethyl, 2-(4-bromobutoxy)ethyl and 2-(2-iodoethoxy)ethyl.

Examples of “carboxyC₁₋₄alkyl” include carboxymethyl, 2-carboxyethyl and 2-carboxypropyl.

Heterocyclic rings are rings containing 1, 2 or 3 ring atoms selected from nitrogen, oxygen and sulphur. “Heterocyclic 5 to 7-membered” rings are pyrrolidinyl, piperidinyl, piperazinyl, homopiperidinyl, homopiperazinyl, thiomorpholinyl , thiopyranyl and morpholinyl. “Heterocyclic 4 to 7-membered” rings include the examples of “heterocyclic 5 to 7-membered” and additionally azetidinyl.

Examples of saturated 3- to 7-membered rings optionally containing 1 or 2 heteroatom groups selected from NH, O, S, SO or SO₂ include cyclopropyl, cyclohexane, cyclopentane, piperidine, pyrrolidine, morpholine, terahydofuran and tetrahydropyran. Examples of saturated 5- to 7-membered rings optionally containing a heteroatom groups selected from NH, O, S, SO or SO₂ include cyclohexane, cyclopentane, piperidine, pyrrolidine, terahydofuran and tetrahydropyran.

Where optional substituents are chosen from “one of more” groups or substituents it is to be understood that this definition includes all substituents being chosen from one of the specified groups or the substituents being chosen from two or more of the specified groups. Preferably “one or more” means “1, 2 or 3” and this is particularly the case when the group or substituent is halo. “One or more” may also mean “1 or 2”.

Compounds of the present invention have been named with the aid of computer software (ACD/Name version 5.09).

Preferred values of z, n, W, t, B, R³, R⁴, R⁵, R⁶, R⁷, R¹² and R¹³ are as follows. Such values may be used where appropriate with any of the definitions, claims or embodiments defined herein.

In one aspect of the invention z is NR⁸.

In one aspect of the invention n is 1. In another aspect n is 0.

In one aspect of the invention W is CR¹R². In a further aspect W is a bond.

In one aspect of the invention t is 0. In another aspect t is 1.

In one aspect of the invention, B is a group selected from aryl, heteroaryl and heterocyclyl where each group is optionally substituted by one or more groups independently selected from nitro, trifluoromethyl, trifluoromethoxy, halo, C₁₋₄alkyl (optionally substituted by one or more halo), C₂₋₄alkynyl, heteroaryl, —OR⁹, cyano, —NR⁹R¹⁰, —CONR⁹R¹⁰ and —NR¹⁹COR¹⁰; or B is C₂₋₄alkenyl or C₂₋₄alkynyl optionally substituted by C₁₋₄alkyl, C₃₋₆cycloalkyl or heterocyclyl. In another aspect B is a group selected from bicyclic aryl or bicyclic heteroaryl where each group is optionally substituted by one or more groups independently selected from nitro, trifluoromethyl, trifluoromethoxy, halo, C₁₋₄alkyl (optionally substituted by one or more halo), C₂₋₄alkynyl, heteroaryl, —OR⁹, cyano, —NR⁹R¹⁰, —CONR⁹R¹⁰ and —NR⁹COR¹⁰; or B is C₂₋₄alkenyl or C2-4alkynyl optionally substituted by C₁₋₄alkyl, C₃₋₆cycloalkyl or heterocyclyl. In another aspect, B is phenyl, naphthyl, pyridyl, quinolinyl, isoquinolinyl, thienopyridyl, 1,8-naphthyridinyl, 2,3-methylenedioxyphenyl, 3,4-methylenedioxyphenyl, 1,6-naphthyridinyl, thienopyrimidinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl or isoindolinyl, where each is optionally substituted by one or more groups independently selected from nitro, trifluoromethyl, trifluoromethoxy, halo, C₁₋₄alkyl (optionally substituted by one or more fluoro), C₂₋₄alkynyl, heteroaryl, —OR⁹, cyano, —NR⁹R¹⁰, —CONR⁹R¹⁰ and —NR⁹COR¹⁰; or B is vinyl or ethynyl optionally substituted by C₁₋₄alkyl. In another aspect B is phenyl, naphthyl, pyridyl, quinolinyl, isoquinolinyl, thieno[2,3-b]pyridyl, thieno[3,2-b]pyridyl, 1,8-naphthyridinyl, 2,3-methylenedioxyphenyl, 3,4-methylenedioxyphenyl, 1,6-naphthyridinyl, thieno[2,3-d]pyrimidinyl or thieno[3,2-d]pyrimidinyl where each is optionally substituted by one or more groups independently selected from trifluoromethyl, trifluoromethoxy, fluoro, chloro, bromo, methyl, isopropyl, ethynyl, cyano, acetamido, propyloxy, isopropyloxy, methoxy, nitro, pyrrolidinylcarbonyl, N-propylcarbamoyl, pyrrolidinyl, piperidinyl, isoxazolyl, pyrazolyl, imidazolyl, oxazolyl, thiazolyl, pyrimidinyl and pyridyl; or B is vinyl or ethynyl optionally substituted by methyl or ethyl. In a further aspect B is quinolin-4-yl, naphthyl, 2-methylquinolin-4-yl, 3-methylnaphthyl, 7-methylquinolin-5-yl, 6-methylquinolin-8-yl, 7-methylisoquinolin-5-yl, 6-methylthieno[2,3-b]pyridyl, 5-methylthieno[3,2-b]pyridyl, 2-methyl-1,8-naphthyridinyl, 2-trifluoromethylquinolin-4-yl, 2-ethynylquinolin-4-yl, 7-chloroquinolin-5-yl, 7-fluoro-2-methylquinolin-4-yl, 2-methyl-N-oxoquinolin-4-yl, 3-methylisoquinolin-1-yl, 5-fluoro-2-methylquinolin-4-yl, 2,6-dimethylpyrid-4-yl, 2,5-dimethylpyridin-4-yl, 2,5-dimethylphenyl, 2,5-difluorophenyl, 2,6-difluoro-3-methylphenyl, 2-chloro-6-fluorophenyl, 5-fluoro-2-methylphenyl, 2,6-difluorophenyl, 2,6-dichlorophenyl, 3,5-dimethylphenyl, 2,3-methylenedioxyphenyl, 3,4-methylenedioxyphenyl, 5-fluoro-2-methylpyridinyl, 1-methylquinolinyl, 7-chloroquinolin-4-yl, 8-chloroquinolin-4-yl, 3-chloro-5-trifluoromethylpyrid-2-yl, 3,5-dichloropyrid-2-yl, 6-chloroquinolin-4-yl, 5-methylthieno[2,3-d]pyrimidin-4-yl, 7-methylthieno[3,2-d]pyrimidin-4-yl, 8-fluoroquinolin-4-yl, 6-fluoroquinolin-4-yl, 2-methylquinolin-4-yl, 6-chloro-2-methylquinolin-4-yl, 1,6-naphthyridin-4-yl, thieno[3,2-b]pyrid-7-yl, 2-chloro-5-fluorophenyl, ethynyl, prop-1-enyl, prop-1-ynyl or but-1-ynyl. In another aspect of the invention B is a group selected from quinolinyl, pyridyl and phenyl where each group is optionally substituted by one or more methyl, trifluoromethyl, trifluoromethoxy, halo or isoxazolyl. In a further aspect B is aryl, heteroaryl or C₂₋₄alkynyl optionally substituted by halo or C₁₋₄alkyl. In another aspect B is 2-methylquinolin-4-yl, 2,5-dimethylphenyl, 2,5-dimethylpyrid-4-yl, phenyl, 3,5-difluorophenyl or prop-1-ynyl. In a further aspect of the invention B is 2-methylquinolin4-yl, 2,5-dimethylphenyl or 2,5-dimethylpyrid-4-yl. In yet another aspect B is 2-methylquinolin-4-yl or 2,5-dimethylphenyl.

In one aspect of the invention R¹ is hydrogen or methyl.

In one aspect of the invention R² is hydrogen or methyl.

In one aspect of the invention R³ is hydrogen, methyl, ethyl, propyl or phenyl. In another aspect R³ is hydrogen or methyl.

In one aspect of the invention Rhu 1 and R³ together with the carbon atoms to which they are attached form a 2,2-dimethylthiomorpholine, piperidine, pyrrolidine, piperazine, morpholine, cyclopentane or cyclohexane ring.

In one aspect of the invention R⁴ is hydrogen or methyl. In another aspect R⁴ is hydrogen.

In one aspect of the invention R³ and R⁴ together form a pyrrolidine ring, a piperidine ring, a tetrahydrofuran ring or a tetrahydropyran ring. In another aspect R³ and R⁴ together form a pyrrolidine ring or a tetrahydro-2H-pyran ring.

In one aspect of the invention R⁵ is hydrogen or methyl.

In one aspect of the invention R³ and R⁵ together with the carbon atoms to which they are attached form a piperidine ring optionally substituted by methyl.

In one aspect of the invention R⁶ is hydrogen or methyl.

In one aspect of the invention R⁷ is hydrogen or a group selected from C₁₋₆alkyl, C₃₋₇cycloalkyl, aryl, heteroaryl or heterocyclyl which group is optionally substituted by heterocyclyl, aryl and heteroaryl; and wherein the group from which R⁷ may be selected is optionally substituted on the group and/or on its optional substituent by one or more substituents independently selected from halo, cyano, C₁₋₄alkyl, —COC₁₋₃alkyl, —SO₂C₁₋₃alkyl, —OR²¹, —NR²¹R²², —CO₂R²¹, —NR²¹COR²², —NR²¹CO₂R²² and —CONR²¹R²². In another aspect R⁷ is hydrogen or a group selected from C₁₋₄alkyl, arylC₁₋₄alkyl, heteroarylC₁₋₄alkyl, heterocyclylC₁₋₄alkyl, aryl, heteroaryl, heterocyclyl and C₃₋₅cycloalkyl which group is optionally substituted by cyano, C₁₋₄alkyl, halo, —OR²¹, —NR²¹R²², —COC₁₋₃alkyl and —SO₂C₁₋₃alkyl. In a further aspect R⁷ is hydrogen or a group selected from C₁₋₄alkyl, tetrahydrofuran, tetrahydropyran, pyrrolidinyl, piperidinyl and morpholinyl optionally substituted by methyl, ethyl, methoxy, ethoxy, fluoro, —COC₁₋₃alkyl or —SO₂C₁₋₃alkyl. In a further aspect R⁷ is selected from hydrogen, methyl, ethyl, propyl, isopropyl, cyclopropyl, butyl, tert-butyl, isobutyl, 1-hydroxyethyl, 2-hydroxyethyl, 3-hydroxypropyl, aminomethyl, 2-cyanoethyl, phenyl, pyridyl, benzyl, 3-methylbenzyl, phenylethyl, 4-chlorophenylethyl, 4-fluorophenylethyl, phenylpropyl, 4-chlorophenylpropyl, 4-fluorophenylpropyl, piperazin-1-ylmethyl, 4-methylpiperazin-1-ylethyl, morpholin-4-ylpropyl, pyrimidin-2-ylethyl, pyrimidin-2-ylpropyl, pyrimidin-2-ylbutyl, 5-fluoropynmidin-2-ylpropyl, imidazol-1-ylpropyl, imidazol-1-ylbutyl, 1,3,4-triazolylpropyl, piperidinyl, carbamoylphenyl, tetrahydro-2H-pyranyl, tetrahydro-2H-pyranylmethyl, pyrid-2-ylmethyl, pyrid-4-ylmethyl, pyrid-3-ylmethyl, piperidin-4-ylmethyl, N-(methylcarbonyl)piperidin-4-yl, N-(tert-butoxycarbonyl)piperidin-4-yl, benzyloxyethyl, N-(tert-butoxycarbonyl)piperidin-4-ylmethyl, (3,4,4-trimethyl-2,5-dioximidazolidin-1-yl)methyl, methoxymethyl, methoxyethyl and N-benzoyl-N-phenylaminomethyl. In one aspect R⁷ is selected from hydrogen, C₁₋₄alkyl, haloC₁₋₄alkyl, hydroxyC₁₋₄alkyl, C₁₋₄alkoxyC₁₋₄alkyl and aryl. In another aspect R⁷ is hydrogen, methyl, hydroxymethyl, isobutyl or phenyl.

In one aspect of the invention R³ and R⁷ together with the carbon atoms to which they are each attached and (CR⁵R⁶)ₙ form a piperidinyl, pyrrolidinyl, piperazine or morpholine ring.

In one aspect of the invention R⁸ is hydrogen.

In one aspect of the invention R⁹ is hydrogen or methyl.

In one aspect of the invention R¹⁰ is hydrogen or methyl.

In one aspect of the invention R¹¹ is methyl.

In one aspect of the invention R¹² is hydrogen or methyl.

In one aspect of the invention R¹³ is hydrogen or methyl.

In one aspect of the invention R¹⁴ is hydrogen, —NR²³R²⁴ or C₁₋₄alkyl (optionally substituted by halo, —OR²³ and —NR²³R²⁴. In one aspect R¹⁴ is hydrogen, methyl or amino.

In one aspect of the invention R¹⁶ is hydrogen or methyl.

In one aspect of the invention R¹⁷ is selected from fluoro, chloro, methyl or methoxy.

In one aspect of the invention R¹⁹ is a group selected from C₁₋₆alkyl, aryl and arylC₁₋₄alkyl where the group is optionally substituted by halo. In another aspect R¹⁹ is a group selected from methyl, phenyl and benzyl where the group is optionally substituted by chloro. In one aspect R¹⁹ is methyl.

In one aspect of the invention R¹⁸ is hydrogen or a group selected from C₁₋₆alkyl, aryl and arylC₁₋₄alkyl which group is optionally substituted by halo. In another aspect R¹⁸ is hydrogen or a group selected from methyl, phenyl and benzyl which group is optionally substituted by chloro.

In one aspect of the invention R²⁰ is hydrogen or methyl.

In one aspect of the invention R²¹ is hydrogen, methyl, ethyl, phenyl or benzyl. In another aspect R²¹ is hydrogen.

In one aspect R²² is hydrogen, methyl, ethyl, phenyl or benzyl. In another aspect R²² is hydrogen or methyl.

In one aspect of the invention R²³ is hydrogen or methyl.

In one aspect of the invention R²⁴ is hydrogen or methyl.

In one aspect of the invention R²⁵ is a group selected from C₁₋₆alkyl, aryl and arylC₁₋₄alkyl which group is optionally substituted by halo. In another aspect R²⁵ is a group selected from methyl, phenyl and benzyl which group is optionally substituted by chloro. In one aspect of the invention R²⁵ is methyl.

A preferred class of compound is of formula (1) wherein:

Y¹ and Y² are both O;

z is NR⁸;

n is 0 or 1;

W is CR¹R² or a bond;

V is a group of formula (A);

t is 1;

B is a group selected from aryl, heteroaryl and heterocyclyl where each group is optionally substituted by one or more groups independently selected from nitro, trifluoromethyl, trifluoromethoxy, halo, C₁₋₄alkyl (optionally substituted by one or more halo), C₂₋₄allynyl, heteroaryl, —OR⁹, cyano, —NR⁹R¹⁰, —CONR⁹R¹⁰ and —NR⁹COR¹⁰; or B is C₂₋₄alkenyl or C₂₋₄alkynyl optionally substituted by C₁₋₄alkyl, C₃₋₆cycloallyl or heterocyclyl.

R¹ and R² are independently hydrogen or methyl;

R³ is hydrogen, methyl, ethyl, propyl or phenyl;

R⁴, R⁵, R⁶, R⁹, R¹⁰, R¹², R²³ and R²⁴ are independently hydrogen or methyl;

R⁷ is hydrogen or a group selected from C₁₋₆alkyl, C₃₋₇cycloalkyl, aryl, heteroaryl or heterocyclyl which group is optionally substituted by heterocyclyl, aryl and heteroaryl; and wherein the group from which R⁷ may be selected is optionally substituted on the group and/or on its optional substituent by one or more substituents independently selected from halo, cyano, C₁₋₄alkyl, —COC₁₋₃alkyl, —SO₂C₁₋₃alkyl, —OR²¹, —NR²¹R²², —CO₂R²¹, —NR²¹ COR²², —NR²¹CO₂R²² and —CONR²¹R²²;

R⁸ is hydrogen;

R¹⁴ is hydrogen, —NR²³R²⁴ or C₁₋₄alkyl (optionally substituted by halo, —OR²³ or —NR²³R²⁴); and

R²¹ and R²² are independently hydrogen, methyl, ethyl, phenyl or benzyl.

Another preferred class of compounds is of formula (1) wherein:

Y¹ and Y² are both O;

z is NR⁸;

n is 0 or 1;

W is CR¹R² or a bond;

V is a group of formula (A);

t is 1;

B is phenyl, naphthyl, pyridyl, quinolinyl, isoquinolinyl, thienopyridyl, 1,8-naphthyridinyl, 2,3-methylenedioxyphenyl, 3,4-methylenedioxyphenyl, 1,6-naphthyridinyl, thienopyrimidinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl or isoindolinyl, where each is optionally substituted by one or more groups independently selected from nitro, trifluoromethyl, trifluoromethoxy, halo, C₁₋₄alkyl (optionally substituted by one or more fluoro), C₂₋₄alkynyl, heteroaryl, —OR⁹, cyano, —NR⁹R¹⁰, —CONR⁹R¹⁰ and —NR⁹COR¹⁰; or B is vinyl or ethynyl optionally substituted by C₁₋₄alkyl;

R¹ and R² are independently hydrogen or methyl;

R³, R⁴, R⁵, R⁶, R⁹, R¹⁰, R¹² and R¹³ are independently hydrogen or methyl; and

R⁷ is hydrogen, C₁₋₄alkyl, haloC₁₋₄alkyl, hydroxyC₁₋₄alkyl, C₁₋₄alkoxyC₁₋₄alkyl or aryl;

R⁸ is hydrogen; and

R¹⁴ is hydrogen, methyl or amino.

Another preferred class of compounds is of formula (1) wherein:

Y¹ and Y² are both O;

z is NR⁸;

n is 0 or 1;

W is CR¹R² or a bond;

V is a group of formula (A);

t is 1;

B is aryl, heteroaryl or C₁₋₄alkynyl optionally substituted by halo or C₁₋₄alkyl;

R¹ and R² are independently hydrogen or methyl;

R³, R⁴, R⁵, R⁶, R¹² and R¹³ are independently hydrogen or methyl; and

R⁷ is hydrogen, C₁₋₄alkyl, haloC₁₋₄alkyl, hydroxyC₁₋₄alkyl, C₁₋₄alkoxyC₁₋₄alkyl or aryl.

R⁸ is hydrogen; and

R¹⁴ is hydrogen, methyl or amino.

Another preferred class of compounds is of formula (1) wherein:

Y¹ and Y² are both O;

z is NR⁸;

n is 0;

W is a bond;

V is a group of formula (A);

t is 1;

B is a group selected from aryl, heteroaryl and heterocyclyl where each group is optionally substituted by one or more groups independently selected from nitro, trifluoromethyl, trifluoromethoxy, halo, C₁₋₄alkyl (optionally substituted by one or more halo), C₂₋₄allynyl, heteroaryl, —OR⁹, cyano, —NR⁹R¹⁰, —CONR⁹R¹⁰ and —NR⁹COR¹⁰; or B is C₂₋₄alkenyl or C₂₋₄alkyl optionally substituted by C₁₋₄alkyl, C₃₋₆cycloalkyl or heterocyclyl;

R³, R⁴, R⁵, R⁶, R⁹, R¹⁰, R¹² and R¹³ are independently hydrogen or methyl; and

R⁷ is hydrogen, C₁₋₄alkyl, haloC₁₋₄alkyl, hydroxyC₁₋₄alkyl, C₁₋₄alkoxyC₁₋₄alkyl or aryl.

R⁸ is hydrogen; and

R¹⁴ is hydrogen, methyl or amino.

Another preferred class of compounds is of formula (1) wherein:

Y¹ and Y² are both O;

z is NR⁸;

n is 0;

W is a bond;

V is a group of formula (A);

t is 1;

B is aryl, heteroaryl or C₁₋₄alkynyl optionally substituted by halo or C₁₋₄alkyl

R¹ and R² are independently hydrogen or methyl;

R³, R⁴, R⁵, R⁶, R¹² and R¹³ are independently hydrogen or methyl; and

R⁷ is hydrogen, C₁₋₄alkyl, haloC₁₋₄alkyl, hydroxyC₁₋₄alkyl, C₁₋₄alkoxyC₁₋₄alkyl or aryl.

R⁸ is hydrogen; and

R¹⁴ is hydrogen, methyl or amino.

In another aspect of the invention, preferred compounds of the invention are any one of:
(R/S)-5-(1-{3-methyl-3-[4-(2-methylquinolin-4-ylmethoxy)phenyl]-2-oxopyrrolidin-1-yl}ethyl)imidazolidine-2,4-dione;
(R/S)-5-{3-methyl-3-[4-(2-methylquinolin-4-ylmethoxy)phenyl]-2-oxopyrrolidin-1-ylmethyl}imidazolidine-2,4-dione;
5-methyl-5-{3-methyl-3-[4-(2-methylquinolin-4-ylmethoxy)phenyl]-2-oxopyrrolidin-1-ylmethyl}imidazolidine-2,4-dione;
5-{3-amino-3-[4-(2-methylquinolin-4-ylmethoxy)phenyl]-2-oxopyrrolidin-1-ylmethyl}imidazolidine-2,4-dione dihydrochloride;
5-[3-(4-benzyloxyphenyl)-3-methyl-2-oxopyrrolidin-1-ylmethyl]imidazolidine-2,4-dione;
5-{3-methyl-3-[4-(2-methylquinolin-4-ylmethoxy)phenyl]-2-oxopyrrolidin-1-ylmethyl}-5-phenylimidazolidine-2,4-dione;
5-isobutyl-5-{3-methyl-3-[4-(2-methylquinolin-4-ylmethoxy)phenyl]-2-oxopyrrolidin-1-ylmethyl}imidazolidine-2,4-dione;
5-[(3-{4-[(2,5-dimethylbenzyl)oxy]phenyl}-3-methyl-2-oxopyrrolidin-1-yl)methyl]imidazolidine-2,4-dione;
5-[(3-{4-[(3,5-difluorobenzyl)oxy]phenyl}-3-methyl-2-oxopyrrolidin-1-yl)methyl]imidazolidine-2,4-dione;
5-({3-[4-(but-2-yn-1-yloxy)phenyl]-3-methyl-2-oxopyrrolidin-1-yl}methyl)imidazolidine-2,4-dione;
5-hydroxymethyl-5-{3-methyl-3-[4-(2-methyl-quinolin-4-ylmethoxy)phenyl]-2-oxo-pyrrolidin-1-ylmethyl}-imidazolidine-2,4-dione;
5-[(3-{4-[(2,5-dimethylbenzyl)oxy]phenyl}-3-methyl-2-oxopyrrolidin-1-yl)methyl]-5-methylimidazolidine-2,4-dione;
5-({3-methyl-3-[4-(1-naphthylmethoxy)phenyl]-2-oxopyrrolidin-1-yl}methyl)imidazolidine-2,4-dione; and
5-({3-amino-3-[4-(1-naphthylmethoxy)phenyl]-2-oxopyrrolidin-1-yl}methyl)imidazolidine-2,4-dione.

In another aspect the present invention provides a process for the preparation of a compound of formula (1) or a pharmaceutically acceptable salt or in vivo hydrolysable ester thereof wherein Y¹ and Y² are both O, z is NR⁸ and R⁸ is hydrogen, which comprises converting a ketone or aldehyde of formula (2) into a hydantoin of formula (1); 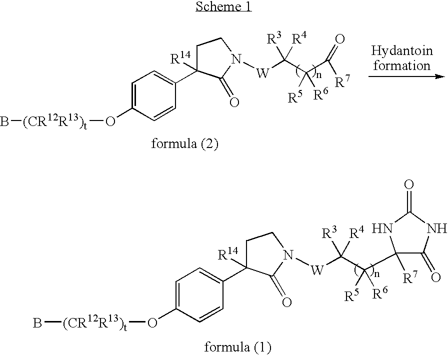
and thereafter if necessary:
i) converting a compound of formula (1) into another compound of formula (1);
ii) removing any protecting groups;
iii) forming a pharmaceutically acceptable salt or in vivo hydrolysable ester.
The hydantoin can be prepared by a number of methods for example:
a) The aldehyde or ketone may be reacted with ammonium carbonate and potassium cyanide in aqueous alcohols using the method of Bucherer and Bergs (Adv. Het. Chem., 1985, 38, 177).
b) The aldehyde or ketone could be first converted to the cyanohydrin and then further reacted with ammonium carbonate (Chem. Rev, 1950, 56, 403).
c) The aldehyde or ketone could be converted to the alpha-amino nitrile and then either reacted with ammonium carbonate or aqueous carbon dioxide or potassium cyanate followed by mineral acid (Chem. Rev, 1950, 56, 403).

A process for the preparation of a ketone or aldehyde of formula (2) comprises converting a compound of formula (3) into a ketone or aldehyde of formula (2): 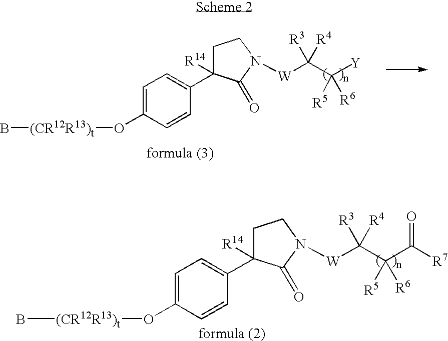
wherein Y is an ester group such as —COOC₁₋₁₀alkyl; a ketal such as 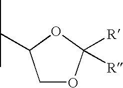
where R′ and R″ are C₁₋₁₀alkyl; an alcohol group such as —CHR⁷OH; or an alkene group such as CR⁷═CH₂.
a) when Y is an ester group so that scheme 2 illustrates the reaction: 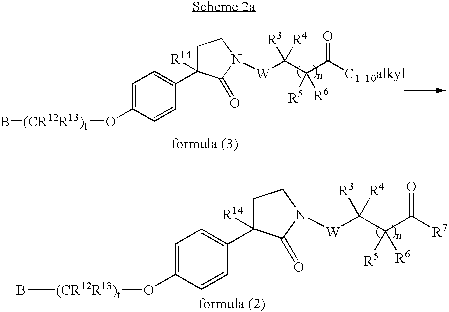
suitable reagents are Grignard reagents to prepare ketones or diisobutylaluminium hydiide in dichloromethane at −78° C. under an argon atmosphere to prepare aldehydes.
b) when Y is a ketal so that scheme 2 illustrates the reaction: 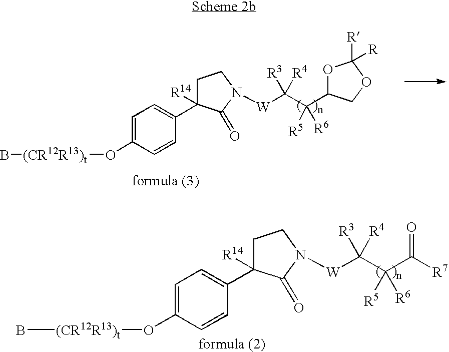
a suitable reagent is an aqueous acid (eg a mineral acid such as hydrochloric acid) to hydrolyse the ketal to the diol (Protective Groups in Organic Synthesis; Theordora Greene and Peter Wuts, Wiley-InterScience), followed by treatment with sodium periodate or osmium tetraoxide to generate the aldehyde. This can be converted directly to the hydantoin as described above, or reacted with Grignard reagents or alkyl lithiums to prepare secondary alcohols, which can be oxidised to the ketones with an oxidising agent.
c) when Y is an alcohol group so that scheme 2 illustrates the reaction: 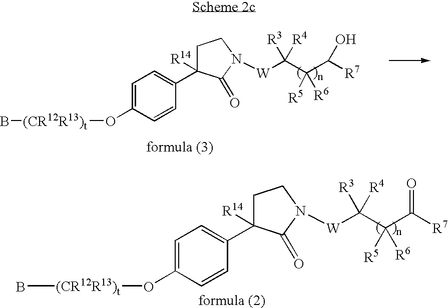
suitable reagents are oxidising agents.
d) when Y is an alkene group so that scheme 2 illustrates the reaction: 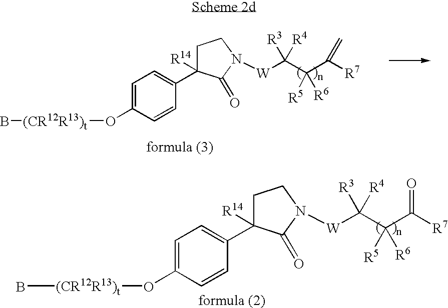
suitable reagents include reagents for ozonolysis, sodium periodate, osmium tetraoxide and ruthenium calalysts with a suitable oxidant.

An alternative to scheme 2a, for the preparation of the aldehyde or ketone of formula (2) from an ester of formula (3) is shown in Scheme 3 which comprises: 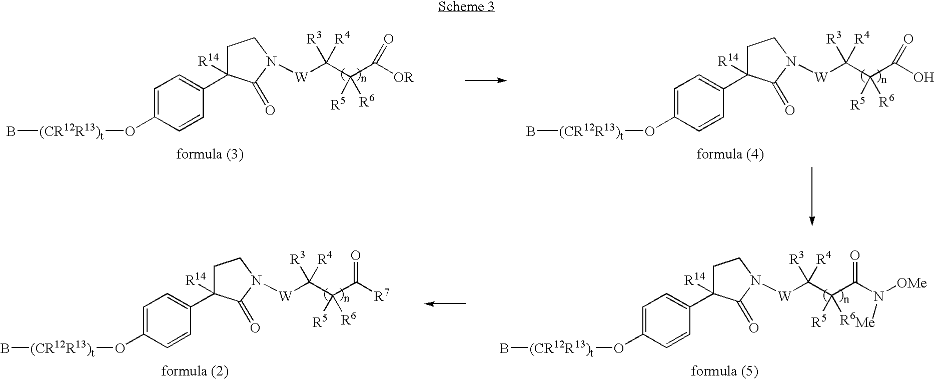
a) reacting the ester of formula (3) with a base such as sodium hydroxide, potassium hydroxide or potassium carbonate in alcohols or aqueous alcohols at room temperature to 100° C. followed by neutralisation with e.g. acetic acid, to give an acid of formula (4);
b) reacting the acid of formula (4) with N, O-dimethlyhydroxylamine hydrochloride under standard amide coupling conditions or by reacting with triphenylphosphine, carbon tetrabromide and triethylamine in dichlormethane for 10 to 60 minutes (Synth. Commun., 1990, 20, 1105), to give an amide of formula (5); and
c) reacting the amide of formula (5) with a reducing agent such as diisobutylaluminium hydride or lithium aluminium hydride to give an aldehyde of formula (2) or reacting with Grignard reagents to give a ketone of formula (2).

A compound of formula (3) may be prepared as shown in Scheme 4; 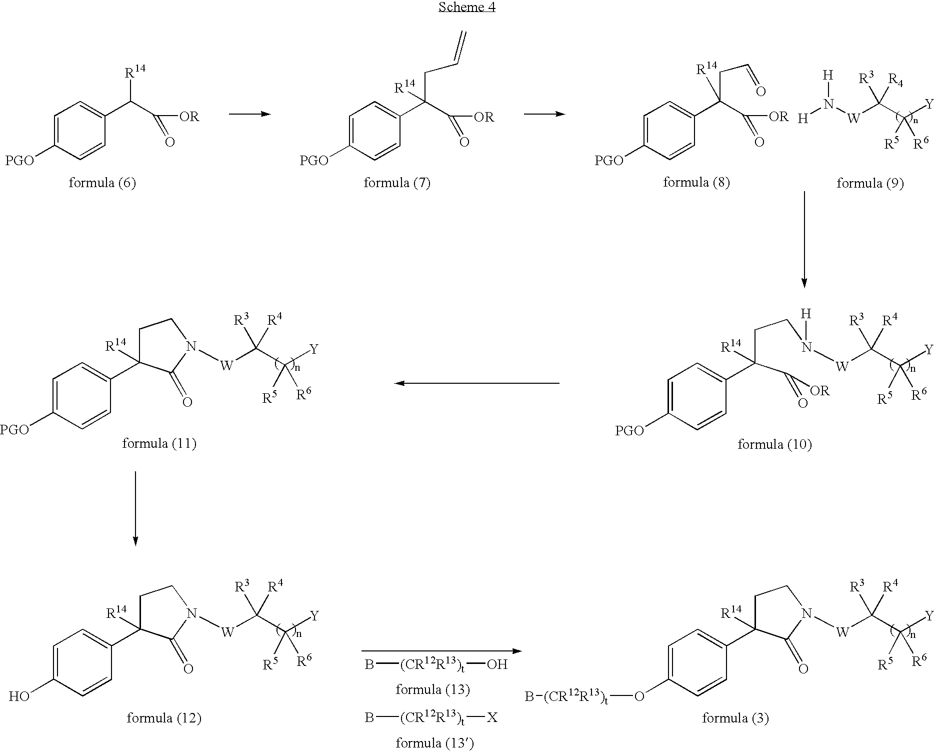
The process of Scheme 4 comprises the steps of:
a) reacting an ester of formula (6), where PG is a protecting group such as benzyl and R is C₁₋₁₀alkyl, with a base such lithium diisopropylamide or lithium bis(trimethylsilyl)amide in tetrahydrofuran at a temperature of −78° C. to 0° C. followed by reaction with allyl bromide for 30 minutes to 2 hours to give an allylated product of formula (7);
b) reacting the allylated product of formula (7) with ozone, until no more starting compound can be observed by thin layer chromatography or high performance liquid chromatography/mass spectrometry followed by reduction of the resultant ozonide with e.g. dimethylsulphide, triphenylphosphine or polymer supported triphenylphosphine to give an aldehyde of formula (8);
c) reacting the aldehyde of formula (8) with an amine or amine salt of formula (9) (where Y is an ester group, a ketal, an alcohol group or an alkene group as defined above) in a solvent such as dichloromethane or dichloroethylene in the presence of a base such as triethylamine or N,N-diisopropylethylamine for 30 minutes to 2 hours before addition of a reducing agent such as sodium triacetoxyborohydride, sodium borohydride or sodium cyanoborohydride and reacted at room temperature for 2 to 24 hours to give an amine of formula (10);
d) cyclisation of the amine of formula (10) by heating in an inert solvent such as toluene to 90-110° C. for 1 to 4 hour to give a lactam of formula (11);
e) removal of the protecting group to give a phenol of formula (12) (if a benzyl protecting group is used this can be removed by treatment with palladium on carbon in the presence of either hydrogen of cyclohexene; for a silyl protecting group, mild acid hydrolysis or treatment with fluoride ion can be used);
f) reacting the phenol of formula (12) with an alcohol of formula (13) under Mitsunobu type conditions or by reaction of the phenol with a halide of formula (13′) by deprotonation with a base such as sodium hydride, lithium bis(trimethylsilyl)amide in a solvent such as dimethylformamide or tetrahydrofuran at 0° C. to 100° C. or deprotonation with caesium carbonate in the presence of tetrabutyl ammonium iodide in dimethylsulphoxide at room temperature to 100° C. to give a compound of formula (3).

A compound of formula (1) can be prepared by removal of protecting groups on the hydantoin directly. The protecting group can be tert-butyloxycarbonyl (BOC), benzyl (Bn) or benzyloxycarbonyl (cbz). These can be removed by treatment with trifluoroacetic acid or hydrogen chloride in dioxane for the former or by treatment with palladium/hydrogen for the latter two.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses metalloproteinases inhibitory activity, and in particular TACE inhibitory activity. This property may be assessed, for example, using the procedure set out below.

### Isolated Enzyme Assays

Matrix Metalloproteinase Family Including for Example MMP13.

Recombinant human proMMP13 may be expressed and purified as described by Knauper et al. [V. Knauper et al., (1996) The Biochemical Journal 271:1544-1550 (1996)]. The purified enzyme can be used to monitor inhibitors of activity as follows: purified proMMP13 is activated using lmM amino phenyl mercuric acid (APMA), 20 hours at 21° C.; the activated MMP13 (11.25 ng per assay) is incubated for 4-5 hours at 35° C. in assay buffer (0.1M Tris-HCl, pH 7.5 containing 0.1M NaCl, 20 mM CaCl₂, 0.02 mM ZnCl and 0.05% (w/v) Brij 35 using the synthetic substrate 7-methoxycoumarin-4-yl)acetyl.Pro.Leu.Gly.Leu.N-3-(2,4-dinitrophenyl)-L2,3-diaminopropionyl.Ala.Arg.NH₂ in the presence or absence of inhibitors. Activity is determined by measuring the fluorescence at λex 328 nm and λem 393 nm. Percent inhibition is calculated as follows: % Inhibition is equal to the [Fluorescence_{plus inhibitor}−Fluorescence_{background}] divided by the [Fluorescence_{minus inhibitor}−Fluorescence_{background}].

A similar protocol can be used for other expressed and purified pro MMPs using substrates and buffers conditions optimal for the particular MMP, for instance as described in C. Graham Knight et al., (1992) FEBS Lett. 296(3):263-266.

Adamalysin Family Including for Example TNF Convertase

The ability of the compounds to inhibit proTNF-α convertase enzyme (TACE) may be assessed using a partially purified, isolated enzyme assay, the enzyme being obtained from the membranes of TBP-1 as described by K. M. Mohler et al., (1994) Nature 370:218-220. The purified enzyme activity and inhibition thereof is determined by incubating the partially purified enzyme in the presence or absence of test compounds using the substrate 4′,5′-Dimethoxy-fluoresceinyl Ser.Pro.Leu.Ala.Gln.Ala.Val.Arg.Ser.Ser.Ser.Arg.Cys(4-(3-succinimid-1-yl)-fluorescein)-NH₂ in assay buffer (50 mM Tris HCl, pH 7.4 containing 0.1% (w/v) Triton X-100 and 2 mM CaCl₂), at 26° C. for 4 hours. The amount of inhibition is determined as for MMP13 except λex 485 nm and λem 538 nm were used. The substrate was synthesised as follows. The peptidic part of the substrate was assembled on Fmoc-NH-Rink-MBHA-polystyrene resin either manually or on an automated peptide synthesiser by standard methods involving the use of Fmoc-amino acids and O-benzotriazol-1-yl-N,N,N′,N′-tetramethyluronium hexafluorophosphate (HBTU) as coupling agent with at least a 4- or 5-fold excess of Fmoc-amino acid and HBTU. Ser¹ and Pro² were double-coupled. The following side chain protection strategy was employed; Ser¹(But), Gln⁵(Trityl), Arg^{8,12}(Pmc or Pbf), Ser^{9,10,11} (Trityl), Cys¹³(Trityl). Following assembly, the N-terminal Fmoc-protecting group was removed by treating the Fmoc-peptidyl-resin with in DMF. The amino-peptidyl-resin so obtained was acylated by treatment for 1.5-2 hours at 70° C. with 1.5-2 equivalents of 4′,5′-dimethoxy-fluorescein-4(5)-carboxylic acid [Khanna & Ullman, (1980) Anal Biochem. 108:156-161) which had been preactivated with diisopropylcarbodiimide and 1-hydroxybenzotriazole in DMF]. The dimethoxyfluoresceinyl-peptide was then simultaneously deprotected and cleaved from the resin by treatment with trifluoroacetic acid containing 5% each of water and triethylsilane. The dimethoxyfluoresceinyl-peptide was isolated by evaporation, trituration with diethyl ether and filtration. The isolated peptide was reacted with 4-(N-maleimido)-fluorescein in DMF containing diisopropylethylamine, the product purified by RP-HPLC and finally isolated by freeze-drying from aqueous acetic acid. The product was characterised by MALDI-TOF MS and amino acid analysis.

The compounds of this invention have been found to be active against TACE (causing greater that 50% inhibition) at less than 10 μM, and in particular 130 nM of compound 6 gave 50% inhibition.

Natural Substrates

The activity of the compounds of the invention as inhibitors of aggrecan degradation may be assayed using methods for example based on the disclosures of E. C. Amer et al., (1998) Osteoarthritis and Cartilage 6:214-228; (1999) Journal of Biological Chemistry, 274 (10), 6594-6601 and the antibodies described therein. The potency of compounds to act as inhibitors against collagenases can be determined as described by T. Cawston and A. Barrett (1979) Anal. Biochem. 99:340-345.

Inhibition of Metalloproteinase Activity in Cell/Tissue Based Activity

Test as an Agent to Inhibit Membrane Sheddases Such as TNF Convertase

The ability of the compounds of this invention to inhibit the cellular processing of TNF-α production may be assessed in THP-1 cells using an ELUSA to detect released TNF essentially as described K. M. Mohler et al., (1994) Nature 370:218-220. In a similar fashion the processing or shedding of other membrane molecules such as those described in N. M. Hooper et al., (1997) Biochem. J. 321:265-279 may be tested using appropriate cell lines and with suitable antibodies to detect the shed protein.

Test as an Agent to Inhibit Cell Based Invasion

The ability of the compound of this invention to inhibit the migration of cells in an invasion assay may be determined as described in A. Albini et al., (1987) Cancer Research 47:3239-3245.

Test as an Agent to Inhibit Whole Blood TNF Sheddase Activity

The ability of the compounds of this invention to inhibit TNF-α production is assessed in a human whole blood assay where LPS is used to stimulate the release of TNF-α. 160 μl of heparinized (10 Units/ml) human blood obtained from volunteers, was added to the plate and incubated with 20 μl of test compound (duplicates), in RPMI1640+bicarbonate, penicillin, streptomycin, glutamine and 1% DMSO, for 30 min at 37° C. in a humidified (5% CO₂/95 %air) incubator, prior to addition of 20 μl LPS (E. coli. 0111:B4; final concentration 10 μg/ml). Each assay includes controls of neat blood incubated with medium alone or LPS (6 wells/plate of each). The plates are then incubated for 6 hours at 37° C. (humidified incubator), centrifuged (2000 rpm for 10 min; 4° C.), plasma harvested (50-100 μl) and stored in 96 well plates at −70° C. before subsequent analysis for TNF-α concentration by ELISA.

Test as an Agent to Inhibit in Vitro Cartilage Degradation

The ability of the compounds of this invention to inhibit the degradation of the aggrecan or collagen components of cartilage can be assessed essentially as described by K. M. Bottomley et al., (1997) Biochem J. 323:483-488.

In Vivo Assessment

Test as an Anti-TNF Agent

The ability of the compounds of this invention as in vivo TNF-α inhibitors is assessed in the rat. Briefly, groups of female Wistar Alderley Park (AP) rats (90-100 g) are dosed with compound (5 rats) or drug vehicle (5 rats) by the appropriate route e.g. peroral (p.o.), intraperitoneal (i.p.), subcutaneous (s.c.) 1 hour prior to lipopolysaccharide (LPS) challenge (30 μg/rat i.v.). Sixty minutes following LPS challenge rats are anaesthetised and a terminal blood sample taken via the posterior vena cavae. Blood is allowed to clot at room temperature for 2 hours and serum samples obtained. These are stored at −20° C. for TNF-α ELISA and compound concentration analysis.

Data analysis by dedicated software calculates for each compound/dose:

$$Percent inhibition of TNF - α = \frac{Mean TNF - α ( Vehicle control ) - Mean TNF - α ( Treated ) ⨯ 100}{Mean TNF - α ( Vehicle control )}$$

Test as an Anti-Arthritic Agent

Activity of a compound as an anti-arthritic is tested in the collagen-induced arthritis (CIA) as defined by D. E. Trentham et al., (1977) J. Exp. Med. 146:857. In this model acid soluble native type II collagen causes polyarthritis in rats when administered in Freunds incomplete adjuvant. Similar conditions can be used to induce arthritis in mice and primates.

Pharmaceutical Compositions

According to a further aspect of the invention there is provided a pharmaceutical composition which comprises a compound of formula (1), or a pharmaceutically acceptable salt or in vivo hydrolysable ester thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository. The composition may also be in a form suitable for inhalation.

In general the above compositions may be prepared in a conventional manner using conventional excipients.

The pharmaceutical compositions of this invention will normally be administered to humans so that, for example, a daily dose of 0.5 to 75 mg/kg body weight (and preferably 0.5 to 30 mg/kg body weight) is received. This daily dose may be given in divided doses as necessary, the precise amount of the compound received and the route of administration depending on the weight, age and sex of the patient being treated and on the particular disease condition being treated according to principles known in the art.

Typically unit dosage forms will contain about 1 mg to 500 mg of a compound of this invention.

Therefore a further aspect of the present invention provides a compound of formula (1), or a pharmaceutically acceptable salt or in vivo hydrolysable ester thereof, as defined hereinbefore, for use in a method of treatment of a warm-blooded animal such as man by therapy. Also provided is a compound of formula (1), or a pharmaceutically acceptable salt or in vivo hydrolysable ester thereof, as defined hereinbefore, for use in a method of treating a disease condition mediated by one or more metalloproteinase enzymes and in particular a disease condition mediated by TNFα. Further provided is a compound of formula (1), or a pharmaceutically acceptable salt or in vivo hydrolysable ester thereof, as defined hereinbefore, for use in a method of treating inflammatory diseases, autoimmune diseases, allergic/atopic diseases, transplant rejection, graft versus host disease, cardiovascular disease, reperfusion injury and malignancy in a warm-blooded animal such as man. In particular a compound of formula (1), or a pharmaceutically acceptable salt or in vivo hydrolysable ester thereof, as defined hereinbefore, is provided for use in a method of treating rheumatoid arthritis, Crohn's disease and psoriasis, and especially rheumatoid arthritis in a warm-blooded animal such as man. A compound of formula (1), or a pharmaceutically acceptable salt or in vivo hydrolysable ester thereof, is also provided for use in a method of treating a respiratory disorder such as asthma or COPD in a warm-blooded animal such as man.

According to an additional aspect of the invention there is provided a compound of formula (1), or a pharmaceutically acceptable salt or in vivo hydrolysable ester thereof, as defined hereinbefore, for use as a medicament. Also provided is a compound of formula (1), or a pharmaceutically acceptable salt or in vivo hydrolysable ester thereof, as defined hereinbefore, for use as a medicament in the treatment of a disease condition mediated by one or more metalloproteinase enzymes and in particular a disease condition mediated by TNF-α. Further provided is a compound of formula (1), or a pharmaceutically acceptable salt or in vivo hydrolysable ester thereof, as defined hereinbefore, for use as a medicament in the treatment of inflammatory diseases, autoimmune diseases, allergic/atopic diseases, transplant rejection, graft versus host disease, cardiovascular disease, reperfusion injury and malignancy in a warm-blooded animal such as man. In particular a compound of formula (1), or a pharmaceutically acceptable salt or in vivo hydrolysable ester thereof, as defined hereinbefore, is provided for use as a medicament in the treatment of rheumatoid arthritis, Crohn's disease and psoriasis, and especially rheumatoid arthritis in a warm-blooded animal such as man. A compound of formula (1), or a pharmaceutically acceptable salt or ii: vivo hydrolysable ester thereof, as defined hereinbefore, is provided for use as a medicament in the treatment of a respiratory disorder such as asthma or COPD in a warm-blooded animal such as man.

According to this aspect of the invention there is provided the use of a compound of formula (1), or a pharmaceutically acceptable salt or in vivo hydrolysable ester thereof, as defined hereinbefore in the manufacture of a medicament for use in the treatment of a disease condition mediated by one or more metalloproteinase enzymes and in particular a disease condition mediated by TNF-α in a warm-blooded animal such as man. Also provided is the use of a compound of formula (1), or a pharmaceutically acceptable salt or in vivo hydrolysable ester thereof, as defined hereinbefore in the manufacture of a medicament for use in the treatment of inflammatory diseases, autoimmune diseases, allergic/atopic diseases, transplant rejection, graft versus host disease, cardiovascular disease, reperfusion injury and malignancy in a warm-blooded animal such as man. In particular the use of a compound of formula (1), or a pharmaceutically acceptable salt or in vivo hydrolysable ester thereof, as defined hereinbefore, is provided in the manufacture of a medicament for use in the treatment of rheumatoid arthritis, Crohn's disease and psoriasis, and especially rheumatoid arthritis in a warm-blooded animal such as man. The use of a compound of formula (1), or a pharmaceutically acceptable salt or in vivo hydrolysable ester thereof, is also provided in the manufacture of a medicament for use in the treatment of a respiratory disorder such as asthma or COPD in a warm-blooded animal such as man.

According to another aspect of the invention there is provided a compound of formula (1), or a pharmaceutically acceptable salt or in vivo hydrolysable ester thereof, as defined hereinbefore for use in the treatment of a disease condition mediated by one or more metalloproteinase enzymes and in particular a disease condition mediated by TNF-α in a warm-blooded animal such as man. Also provided is a compound of formula (1), or a pharmaceutically acceptable salt or in vivo hydrolysable ester thereof, as defined hereinbefore for use in the treatment of inflammatory diseases, autoimmune diseases, allergic/atopic diseases, transplant rejection, graft versus host disease, cardiovascular disease, reperfusion injury and malignancy in a warm-blooded animal such as man. In particular a compound of formula (1), or a pharmaceutically acceptable salt or in vivo hydrolysable ester thereof, as defined hereinbefore, is provided for use in the treatment of rheumatoid arthritis, Crohn's disease and psoriasis, and especially rheumatoid arthritis in a warm-blooded animal such as man. A compound of formula (1), or a pharmaceutically acceptable salt or in vivo hydrolysable ester thereof, is also provided for use in the treatment of a respiratory disorder such as asthma or COPD in a warm-blooded animal such as man.

According to a further feature of this aspect of the invention there is provided a method of producing a metalloproteinase inhibitory effect in a warm-blooded animal, such as man, in need of such treatment which comprises administering to said animal an effective amount of a compound of formula (1).

According to a further feature of this aspect of the invention there is provided a method of producing a TACE inhibitory effect in a warm-blooded animal, such as man, in need of such treatment which comprises administering to said animal an effective amount of a compound of formula (1). According to this further feature of this aspect of the invention there is provided a method of treating autoimmune disease, allergic/atopic diseases, transplant rejection, graft versus host disease, cardiovascular disease, reperfusion injury and malignancy in a warm-blooded animal, such as man, in need of such treatment which comprises administering to said animal an effective amount of a compound of formula (1). Also provided is a method of treating rheumatoid arthritis, Crohn's disease and psoriasis, and especially rheumatoid arthritis in a warm-blooded animal, such as man, in need of such treatment which comprises administering to said animal an effective amount of a compound of formula (1). Further provided is a method of treating a respiratory disorder such as asthma or COPD in a warm-blooded animal, such as man, in need of such treatment which comprises administering to said animal an effective amount of a compound of formula (1).

In addition to their use in therapeutic medicine, the compounds of formula (1) and their pharmaceutically acceptable salts are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

The compounds of this invention may be used in combination with other drugs and therapies used in the treatment of various immunological, inflammatory or malignant disease states which would benefit from the inhibition of TACE.

If formulated as a fixed dose such combination products employ the compounds of this invention within the dosage range described herein and the other pharmaceutically-active agent within its approved dosage range. Sequential use is contemplated when a combination formulation is inappropriate.

### EXAMPLES

The invention will now be illustrated by the following non-limiting examples in which, unless stated otherwise:
(i) temperatures are given in degrees Celsius (° C.); operations were carried out at room or ambient temperature, that is, at a temperature in the range of 18-25° C.;
(ii) organic solutions were dried over anhydrous magnesium sulphate; evaporation of solvent was carried out using a rotary evaporator under reduced pressure (600-4000 Pascals; 4.5-30 mm Hg) with a bath temperature of up to 60° C.;
(iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Clamorgan, UK. Where Plashmaster II is referred to, this means a UV driven automated chromatography unit supplied by Jones;
(iv) in general, the course of reactions was followed by TLC and reaction times are given for illustration only;
(v) yields, when given, are for illustration only and are not necessarily those which can be obtained by diligent process development; preparations were repeated if more material was required;
(vi) when given, ¹H NMR data is quoted and is in the form of delta values for major diagnostic protons, given in parts per million (ppm) relative to tetramethylsilane (TMS) as an internal standard, determined at 400 MHz using CDCl₃ as the solvent unless otherwise stated; coupling constants (J) are given in Hz;
(vii) chemical symbols have their usual meanings; SI units and symbols are used;
(viii) solvent ratios are given in percentage by volume;
(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion—(M+M)⁺;
(x) LCMS (liquid chromatography mass spectrometry) characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray (ES); where values for m/z are given, generally only ionas which indicate the parent mass are reported, and unless otherwise stated the mass ion quoted is the positive mass ion—(M+H)⁺ and
(xi) the following abbreviations are used:
   min minute(s);
   h hour(s);
   d day(s);
   DMSO dimethyl sulphoxide;
   DMP N-dimethylformamide;
   DCM dichloromethane;
   NMP N-methylpyrrolidinone;
   DIAD di-isopropylazodicarboxylate;
   LHMDS or LiHMDS lithium bis(trimethylsilyl)amide;
   MeOH methanol;
   RT room temperature;
   TFA trifluoroacetic acid;
   EtOH ethanol;
   EtOAc ethyl acetate;
   TBF tetrahydrofuran;
   DIBAL di-isobutylaluminium hydride;
   NMO 4-methylmorpholine N-oxide; and
   TPAP tetra-n-propylammonium perruthenate (VII)

### Example 1

(R/S)-5-(1-{3-Methyl-3-[4-(2-methylquinolin-4-ylmethoxy)phenyl]-2-oxopyrrolidin-1-yl}ethyl)imidazolidine-2,4-dione

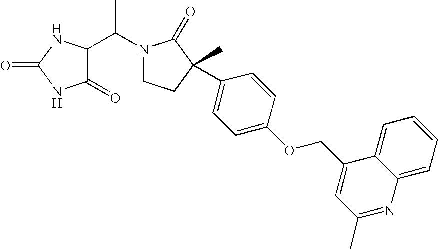

To a stirred solution of 2-{3-methyl-3-[4-(2-methylquinolin-4-ylmethoxy)phenyl]-2-oxo-pyrrolidin-1-yl}propionaldehyde (540 mg, 1.34 mmol) in EtOH (5 ml) and water (5 ml) was added ammonium carbonate (770 mg, 8.0 mmol) and potassium cyanide (174 mg, 2.68 mmol). The mixture was heated to reflux for 1.5 h before addition of a further portion of ammonium carbonate (300 mg, 3.1 mmol). Heating was continued for 1 h and the solution left to stand at RT for 40 h. The solution was reheated to reflux for 3 h, then evaporated under reduced pressure to give a yellow solid. The residue was partitioned between DCM (30 ml) and water (30 ml). The aqueous phase was extracted with DCM (20 ml) and the combined organic phases were dried (Na₂SO₄) and evaporated. The crude product was purified by chromatography (lashmaster II, 20 g silica bond elute, eluent 2% MeOH/DCM) to give the product, as a mixture of 4 diastereoisomers, as a white foam (200 mg, 0.42 mmol); MS: 473.

The starting material 2-{3-methyl-3-[4-(2-methylquinolin-4-ylmethoxy)phenyl]-2-oxo-pyrrolidin-1-yl}propionaldehyde was prepared as follows:
i) To a solution of methyl (R)-2-[3-(4-hydroxyphenyl)-3-methyl-2-oxopyrrolidin-1-yl]propionoate§ (725 mg, 2.62 mmol) in DMSO (30 ml) was added 4-chloromethyl-2-methylquinoline† (500 mg, 2.62 mmol), caesium carbonate (1.7 g, 5.2 mmol) and tetra-n-butylammonium iodide (1.0 g, 2.6 mmol). The resultant solution was stirred at 60° C. for 75 min. The reaction mixture was allowed to cool then diluted with EtOAc (200 ml) and washed with brine (3×100 ml). The organic phase was dried (Na₂SO₄), evaporated and purified by chromatography (Flashmaster II, 50 g silica bond elute, eluent 50→100% EtOAc/isohexane) to give methyl (R)-2-{3-methyl-3-[4-(2-methylquinolin-4-ylmethoxy)phenyl]-2-oxopyrrolidin-1-yl}propionoate (780 mg, 1.8 mmol) as an oil; NMR 1.43 (d, 3H), 1.55 (s, 3H), 2.21 (m, 1H), 2.41 (m, 1H), 2.75 (s, 3H), 3.31 (m, 1H), 3.45 (m, 1H), 3.74 (s, 3H), 4.93 (q, 1H), 5.48 (s, 2H), 6.99 (d, 2H), 7.36 (d, 2H), 7.45 (s, 1H), 7.52 (m, 1H), 7.71 (m, 1H), 7.92 (d, 1H), 8.07 (d, 1H); MS 433.
   § The synthesis of methyl (R)-2-[3-(4-hydroxyphenyl)-3-methyl-2-oxopyrrolidin-1-yl]propionoate has been described in WO99/18974 and has CAS Registry number 223406-12-0.
   † The synthesis of the 4-chloromethyl-2-methylquinoline has been described in WO99/65867 and has CAS Registry number 288399-19-9.
ii) Methyl (R)-2-{3-methyl-3-[4-(2-methylquinolin-4-ylmethoxy)phenyl]-2-oxopyrrolidin-1-yl}propionoate (780 mg, 1.8 mmol) was azeotroped with toluene, dissolved in DCM (10 ml) and the solution cooled to −78° C. To this was added a solution of DIBAL (1.0M in DCM, 3.6 mmol, 3.6 ml) dropwise over 10 min. The solution was stirred at −78° C. for 2 h, before quenching with saturated ammonium chloride solution and allowing to warm to RT. The solution was then diluted with water (20 ml) and DCM (20 ml) and the aqueous phase extracted with DCM (3×30 ml). The combined organic layers were dried (Na₂SO₄), concentrated and purified by chromatography (Flashmaster II, 20 g silica bond elute, eluent 50→100% EtOAc/isohexane) to give 2-{3-methyl-3-[4-(2-methylquinolin-4-ylmethoxy)phenyl]-2-oxopyrrolidin-1-yl}propionaldehyde as a 2:1 mixture of diastereoisomers (540 mg, 1.34 mmol); NMR 1.37 (d, 3H, major isomer), 1.40 (d, 3H, minor isomer), 1.56 (s, 3H, minor isomer), 1.59 (s, 3H, major isomer), 2.22-2.28 (m, 1H), 2.45-2.51 (m, 1H), 2.75 (s, 3H), 3.26-3.36 (m, 2H), 4.71 (q, 1H), 5.49 (s, 2H), 7.00 (d, 2H, minor isomer), 7.01 (d, 2H, major isomer), 7.36 (d, 2H, major isomer), 7.40 (d, 2H, minor isomer), 7.45 (s, 1H), 7.53 (m, 1H), 7.71 (m, 1H), 7.92 (d, 1H), 8.07 (d, 1H); MS: 403.

Alternatively (RIS)-5-(1-{3-methyl-3-[4-(2-methylquinolin-4-ylmethoxy)phenyl]-2-oxopyrrolidin-1-yl}ethyl)imidazolidine-2,4-dione may be prepared as follows: To a stirred solution of 2-{3-methyl-3-[4-(2-methylquinolin-4-ylmethoxy)phenyl]-2-oxopyrrolidin-1-yl}propionaldehyde (100 mg, 0.25 mmol) in EtOH (3 ml) and water (3 ml) was added ammonium carbonate (150 mg, 1.5 mmol) and potassium cyanide (33 mg, 0.5 mmol). The mixture was heated to reflux for 4 h. The solution was left to stand at RT overnight then heated at reflux for 5 h and again stood at RT for 3 d. The solution was evaporated under reduced pressure to give a yellow solid. The residue was partitioned between EtOAc (30 ml) and brine (30 ml). The aqueous phase was extracted with EtOAc (30 ml) and the combined organic phases dried (Na₂SO₄) and evaporated. The crude product was purified by chromatography (Flashmaster II, 20 g silica bond elute, eluent 3% MeOH/DCM) to give the product, as a mixture of 2 diasteoisomers, as a white foam (19 mg, 0.04 mmol); MS: 473.

The starting material 2-{3-methyl-3-[4-(2-methylquinolin-4-ylmethoxy)phenyl]-2-oxopyrrolidin-1-yl}propionaldehyde was prepared as follows:
i) Methyl 2-{3-methyl-3-[4-(2-methylquinolin-4-ylmethoxy)phenyl]-2-oxopyrrolidin-1-yl}propionoate (330 mg, 0.76 mmol) [J. Med. Chem., 2002, 45, 4954.] was dissolved in THF (6 ml). To this was added a solution of lithium borohydride (2.0 M in THF, 1.68 mmol, 0.85 ml). The solution was stirred at RT for 1 h, before quenching with saturated ammonium chloride solution. The solution was then diluted with DCM (20 ml) and the aqueous phase extracted with DCM (10 ml). The combined organic layers were dried (Na₂SO₄), concentrated and purified by chromatography (Flashmaster II, 20 g silica bond elute, eluent 50→100% EtOAc/isohexane) to give 1-(2-hydroxy-1-methylethyl)-3-methyl-3-[4-(2-methylquinolin-4-ylmethoxy)phenyl]pyrrolidin-2-one as a single diastereoisomer (100 mg, 0.25 mmol); NMR (CDCl₃) 1.19 (d, 3H), 1.53 (s, 3H), 2.17 (m, 1H), 2.42 (m, 1H), 2.69 (m, 1H) 2.75 (s, 3H), 3.28 (m, 1H), 3.40 (m, 1H) 3.64 (m, 1H) 3.75 (m, 1H), 4.15 (m, 1H), 5.48 (s, 2H), 7.00 (d, 2H), 7.35 (d, 2H), 7.43 (s, 1H), 7.53 (m, 1H), 7.71 (m, 1H), 7.92 (d, 1H), 8.07 (d, 1H); MS: 405.
ii) 1-(2-Hydroxy-1-methylethyl)-3-methyl-3-[4-(2-methylquinolin-4-ylmethoxy)phenyl]pyrrolidin-2-one (100 mg, 0.25 mmol) was dissolved in DCM (2.5 ml). To this was added a solution of Dess-Martin reagent (15% w/v in DCM, 0.7 ml). The solution was stirred at RT for 3 h and the reaction mixture then diluted with EtOAc (40 ml), washed with brine (20 ml), dried (Na₂SO₄) and evaporated. The resultant product was used in the final step without purification; MS: 403.

### Example 2

(R/S)-5-{3-Methyl-3-[4-(2-methyiquinolin-4-ylmethoxy)phenyl]-2-oxopyrrolidin-1-ylmethyl}imnidazolidine-2,4-dione

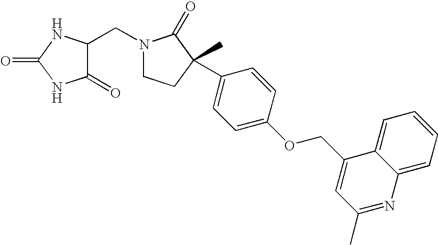

To a stirred solution of {3-methyl-3-[4-(2-methylquinolin-4-ylmethoxy)phenyl]-2-oxopyrrolidin-1-yl}acetaldehyde (450 mg, 1.16 mmol) in EtOH (5 ml) and water (5 ml) was added ammonium carbonate (668 mg, 7.0 mmol) and potassium cyanide (151 mg, 2.3 mmol). The mixture was heated to reflux for 3 h before addition of a further portion of ammonium carbonate (300 mg, 3,1 mmol). Heating was continued for 1 h and the solution allowed to cool and evaporated. The residue was partitioned between DCM (30 ml) and water (30 ml). The aqueous phase was extracted with DCM (30 ml) and the combined organic phases dried (Na₂SO₄) and evaporated. The crude product was purified by chromatography (Flashmaster II, 20 g silica bond elute, eluent 2%→5% MeOH in DCM) to give the product, as a mixture of 2 diasteoisomers, as a white foam (130 mg, 0.28 mmol); MS: 457.

The starting material {3-methyl-3-[4-(2-methylquinolin-4-ylmethoxy)phenyl]-2-oxopyrrolidin-1-yl}acetaldehyde was prepared as follows:
i) To a solution of methyl 2-(4-benzyloxyphenyl)-2-methyl-4-oxobutanoate‡ (3.71 g, 11.9 mmol) in 1,2-dichloroethane was added methyl glycinate hydrochloride (1.6 g, 12.7 mmol) and diisopropylethylamine (2.3 ml, 13.2 mmol). The resultant solution was stirred at RT for 90 min before addition of sodium triacetoxyborohydride (3.3 g, 15.5 mmol). The reaction mixture was stirred for a further 2 h, before addition of DCM (150 ml) and brine (150 ml). The aqueous phase was extracted with DCM (150 ml). The combined organic phases were dried (Na₂SO₄) and evaporated. The resultant oil was dissolved in toluene (50 ml) and heated to 90° C. for 1 h, allowed to cool, evaporated and purified by chromatography (Flashmaster II, 100 g silica bond elute, eluent 20% EtOAc/isohexane) to give methyl [3-(4-benzyloxyphenyl)-3-methyl-2-oxopyrrolidin-1-yl]acetate (2.18 g, 6.2 mmol) as a white solid; NMR 1.55 (s, 3H), 2.19 (m, 1H), 2.43 (m, 1H), 3.41 (m, 2H), 3.73 (s, 3H), 4.13 (s, 2H), 5.04 (s, 2H), 6.93 (d, 2H) 7.29-7.43 (m, 7H); MS 354.
   ‡ The synthesis of methyl 2-(4-benzyloxyphenyl)-2-methyl-4-oxobutanoate has been described in J. Med. Chem., 2002, 45, 4954., WO99/18974 and has CAS Registry number 223406-00-6.
ii) To a solution of methyl [3-(4-benzyloxyphenyl)-3-methyl-2-oxopyrrolidin-1-yl]acetate (2.18 g, 6.2 mmol) in EtOH (50 ml) was added cyclohexene (6.3 ml, 62 mmol) and 10% Pd/C (1.0 g). The reaction mixture was heated under reflux for 1 h. The reaction mixture was allowed to cool and evaporated to give methyl [3-(4-hydroxyphenyl)-3-methyl-2-oxopyrrolidin-1-yl]acetate as an oil (1.6 g, 60.8 mmol); NMR 1.55 (s, 3H), 2.19 (m, 1H), 2.42 (m, 1H), 3.44 (m, 2H), 3.74 (s, 3H), 4.13 (s, 2H), 6.74 (d, 2H), 7.24 (d, 2H). MS 264.
iii) To a solution of methyl [3-(4-hydroxyphenyl)-3-methyl-2-oxopyrrolidin-1-yl]acetate (1.0 g, 3.8 mmol) in DMSO (30 ml) was added 4-chloromethyl-2-methylquinoline† (725 mg, 3.8 mmol), caesium carbonate (2.48 g, 7.6 mmol) and tetra-n-butylammonium iodide (1.4 g, 3.8 mmol). The resultant solution was stirred at 60° C. for 90 min. The reaction mixture was allowed to cool then diluted with EtOAc (200 ml) and washed with brine (3×100 ml). The organic phase was dried (Na₂SO₄), evaporated and purified by chromatography (Flashmaster II, 50 g silica bond elute, eluent 50→100% EtOAc/isohexane) to give methyl {3-methyl-3-[4-(2-methylquinolin-4-ylmethoxy)phenyl]-2-oxopyrrolidin-1-yl}acetate (1.0 g, 2.4 mmol) as an oil; NMR 1.57 (s, 3H), 2.21 (m, 1H), 2.44 (m, 1H), 2.75 (s, 3H), 3.44 (m, 2H), 3.74 (s, 3H), 4.15 (s, 2H), 5.49 (s, 2H), 7.00 (d, 2H), 7.39 (d, 2H), 7.47 (s, 1H), 7.53 (m, 1H), 7.71 (m, 1H), 7.92 (d, 1H), 8.07 (d, 1H); MS 419.
   † The synthesis of the 4-chloromethyl-2-methylquinoline has been described in WO99/65867 and has CAS Registry number 288399-19-9.
iv) Methyl {3-methyl-3-[4-(2-methylquinoln-4-ylmethoxy)pheny]-1-yl}acetate (500 mg, 1.16 mmol) was azeotroped with toluene and dissolved in DCM (6 ml) and the solution cooled to −78° C. To this was added a solution of DIBAL (1.0M in DCM, 2.3 mmol, 2.3 ml) dropwise over 10 min. The solution was stirred at −78° C. for 1 h, before quenching with saturated ammonium chloride solution and allowing to warm to RT. The solution was then diluted with water (10 ml) and DCM (10 ml) and the aqueous phase extracted with DCM (3×30 ml). The organic phase was dried (Na₂SO₄), and evaporated to give the crude aldehyde which was used without further purification; MS: 489.

### Example 3

5-Methyl-5-{3-methyl-3-[4-(2-methylquinolin-4-ylmethoxy)phenyl]-2-oxopyrrolidin-1-ylmethyl}imidazolidine-2,4-dione

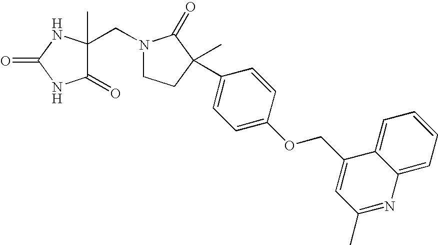

To a stirred solution of 3-methyl-3-[4-(2-methylquinolin-4-ylmethoxy)phenyl]-1-(2-oxopropyl)pyrrolidin-2-one (163 mg, 0.41 mmol) in EtOH (2 ml) and water (2 ml) was added ammonium carbonate (250 mg, 2.6 mmol) and potassium cyanide (55 mg, 0.85 mmol). The mixture was heated to 60° C. for 2.5 h and then 16 h at RT. Silica gel (2 g) was added and the suspension evaporated. The resultant powder was applied to the top of a 10 g bond elute and purified on a Flashmaster II eluting with 0%→10% EtOH in DCM) to give the product, as a mixture of 2 diasteoisomers, as a white foam (99 mg, 0.21 mmol); NMR 1.23 (s, 1.5H), 1.24 (s, 1.5H), 1.376 (s, 1.5H), 1.378 (s, 1.5H), 2.07 (m, 1H), 2.25 (m, 1H), 2.67 (s, 3H), 3.47 (ABq, 1H), 3.68 (d, 0.5H), 5.58 (s, 1H), 5.59 (s, 1H), 7.06 (d, 1H), 7.09 (d, 1H), 7.29 (d, 1H), 7.31 (d, 1H), 7.56 (s, 1H), 7.59 (m, 1H), 7.75 (m, 1H), 7.96 (s, 1H), 8.00 (d, 1H), 8.10 (d, 1H), 10.67 (s, 0.5H), 10.68 (s, 0.5H); MS: 473.

The starting material 3-methyl-3-[4-(2-methylquinolin-4-ylmethoxy)phenyl]-1-(2-oxopropyl)pyrrolidin-2-one was prepared as follows:
i) To a solution of methyl 2-(4-benzyloxyphenyl)-2-methyl-4-oxobutanoate (521 mg, 1.67 mmol) in 1,2-dichloroethane (10 ml) was added 2-amino-1-propanol (0.18 ml, 2.33 mmol). The resultant solution was stirred at RT for 1 h before addition of sodium triacetoxyborohydride (496 mg, 2.34 mmol ). The reaction mixture was stirred for a further 1 h and stood at RT for 72 h before addition of DCM (20 ml) and brine (20 ml). The organic phase was dried (Na₂SO₄) and evaporated. The resultant oil was dissolved in toluene (20 ml) and heated to 90° C. for 2 h, allowed to cool and evaporated. The resultant oil was dissolved in EtOH (10 ml) and placed under an argon atmosphere. Cyclohexene (1.2 ml, 17 mmol) and 10% palladium on charcoal (200 mg) were added and the resultant mixture heated to reflux for 2 h. The reaction mixture was allowed to cool, filtered and evaporated to an oil (440 mg). The crude product was dissolved in DMSO (4 ml). To this caesium carbonate (1.1 g, 3.38 mmol), tetra-n-butylammonium iodide (620 mg, 1.68 mmol) and 4-chloromethyl-2-methylquinoline (333 mg, 1.74 mmol) were added and the mixture heated to 60° C. for 45 min. The reaction mixture was partitioned between EtOAc (20 ml) and brine (20 ml). The organic phase was washed with brine (2×20 ml), dried and evaporated. The crude product was purified by chromatography (Elashmaster II, 20 g silica bond elute, eluent 100% EtOAc) to give 1-(2-hydroxypropyl)-3-methyl-3-[4-(2-methylquinolin-4-ylmethoxy)phenyl]pyrrolidin-2-one as an oil (475 mg); MS: 405.
ii) To a solution of 1-(2-hydroxypropyl)-3-methyl-3-[4-(2-methylquinolin-4-ylmethoxy)phenyl]pyrrolidin-2-one in anhydrous DCM (7 ml) was added NMO (240 mg, 1.8 mmol) and 4A molecular sieves (660 mg). The reaction mixture was stirred for 10 min before addition of TPAP (22 mg, 0.06 mmol), stirring was continued for 20 min and the reaction mixture was poured onto a 5 g Silica bond elute and washed with DCM/MeOH (1:1). The solvent was evaporated to give the crude product which was purified by chromatography (Flashmaster II, eluent 100% EtOAc) to give 3-methyl-3-[4-(2-methylquinolin-4-ylmethoxy)phenyl]-1-(2-oxopropyl)pyrrolidin-2-one as an oil (130 mg, 0.32 mmol); NMR (400 MHz, DMSO), 1.43 (s, 3H), 2.10 (s, 3H), 2.13 (m, 1H), 2.31 (m, 1H), 2.67 (s, 3H), 4.17 (ABq, 2H), 5.58 (s, 2H), 7.09 (d, 2H), 7.37 (d, 2H), 7.56 (s, 1H), 7.59 (m, 1H), 7.74 (m, 1H), 7.97 (d, 1H), 8.11 (d, 1H).

### Example 4

5-{3-Amino-3-[4-(2-methylquinolin-4-ylmethoxy)phenyl]-2-oxopyrrolidin-1-ylmethyl}imidazolidine-2,4-dione dihydrochloride

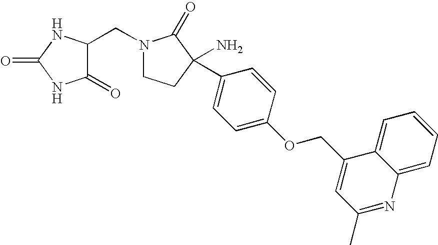

To a stirred solution of acetyl chloride (0.5 ml) in MeOH (5 ml) was added tert-butyl {1-(2,5-dioxoimidazolidin-4-ylmethyl)-3-[4-(2-methylquinolin-4-ylmethoxy)phenyl]-2-oxopyrrolidin-3-yl}carbamate (183 mg, 0.33 mmol). The reaction was stirred at RT for 90 min during which time a white precipitate formed. The reaction mixture was filtered to give a white crystalline solid (90 mg, 0.17 mmol) as a mixture of diastereoisomers; MS: 460. The mother liquors were evaporated to give a further 60 mg of product as an off white solid. 5- {3-Amino-3-[4-(2-methylquinolin-4-ylmethoxy)phenyl]-2-oxopyrrolidin-1-ylmethyl}imidazolidine-2,4-dione dihydrochloride (50 mg) was separated by chiral chromatography (instrument: Gilson, column: Merck 50 mm 20 μm Chiralcel OJ, eluent EtOH/MeOH/TEA 50/50/0.5 at 35 ml/min) to give 4 isomers as the free base, isomer A (8 mg, 79% purity), MS: 460; isomer B (11 mg, 64% purity), MS: 460; isomer C (10 mg, 63% purity) MS: 460 and isomer D (10 mg, 75% purity) MS: 460.

The starting material tert-butyl {1-(2,5-dioxoimidazolidin-4-ylmethyl)-3-[4-(2-methylquinolin-4-ylmethoxy)phenyl]-2-oxo-pyrrolidin-3-yl}carbamate was prepared as follows:
i) To a solution of methyl 2-(4-benzyloxyphenyl)-2-tert-butoxycarbonylamino-4-oxobutanoate (CAS Registry number 223407-41-8) (1.15 g, 2.8 mmol) in 1,2-dichloroethane (15 ml) was added methyl glycinate hydrochloride (390 mg, 3.1 mmol) and diisopropylethylamine (0.54 ml, 0.31 mmol). The resultant solution was stirred at RT for 60 min before addition of sodium triacetoxyborohydride (770 mg, 3.6 mmol). The reaction mixture was stirred for a further 2 h, before addition of DCM (35 ml) and brine (50 ml). The aqueous phase was extracted with DCM (50 ml). The combined organic phases were dried (Na₂SO₄) and evaporated. The resultant oil was dissolved in toluene (30 ml) and heated to 90° C. for 90 min, allowed to cool, evaporated and purified by chromatography (Flashmaster II, 50 g silica bond elute, eluent 20% to 80% EtOAc/isohexane) to give methyl 3-(4-benzyloxyphenyl)-3-tert-butoxycarbonylamino-2-oxopyrrolidin-1-ylacetate (2.18 g, 6.2 mmol) as a colourless oil; NMR (400 MHz, CDCl₃) 1.40 (br. s, 9H), 2.87 (br. s, 2H), 3.38-3.51 (m, 2H), 3.68 (s, 3H), 3.90 (d, 1H), 4.36 (br.d, 1H), 5.05 (s, 2H), 5.50 (br. s, 1H), 6.95 (d, 2H), 7.31-7.45 (m, 7H).
ii) To a solution of methyl 3-(4-benzyloxyphenyl)-3-tert-butoxycarbonylamino-2-oxopyrrolidin-1-ylacetate (800 mg, 1.8 mmol) in EtOH (25 ml) was added cyclohexene (1.8 ml, 18 mmol) and 10% Pd/C (400 mg). The reaction mixture was heated under reflux for 80 min. The reaction mixture was allowed to cool and evaporated to give methyl [3-tert-butoxycarbonylamino-3-(4-hydroxyphenyl)-2-oxopyrrolidin-1-yl]-acetate as white foam (660 mg, 1.8 mmol); NMR (400 MHz CDCl₃) 1.40 (s, 9H), 2.86 (br. s, 2H), 3.42-3.53 (m, 2H), 3.48 (s, 3H), 3.90 (m, 1H), 4.34 (br. d, 1H), 5.56 (br. s, 1H), 6.42 (br. s, 1H), 6.67 (d, 2H), 7.29 (d, 2H).
iii) To a solution of methyl [3-tert-butoxycarbonylamino-3-(4-hydroxyphenyl)-2-oxopyrrolidin-1-yl]acetate (600 mg, 1.6 mmol) in DMSO (15 ml) was added 4-chloromethyl-2-methylquinoline (320 mg, 1.7 mmol), caesium carbonate (1.08 g, 3.3 mmol) and tetra-n-butylammonium iodide (610 mg, 1.65 mmol). The resultant solution was stirred at 60° C. for 70 min. The reaction mixture was allowed to cool then diluted with EtOAc (90 ml) and washed with brine (3×45 ml). The organic phase was dried (Na₂SO₄), evaporated and purified by chromatography (Flashmaster II, 50 g silica bond elute, eluent 40→80% EtOAc/isohexane) to give methyl {3-tert-butoxycarbonylamino-3-[4-(2-methylquinolin-4-ylmethoxy)phenyl]-2-oxopyrrolidin-1-yl}acetate (525 mg, 1.0 mmol) as an oil; NMR (400 MHz, CDCl₃) 1.41 (br. s, 9H), 2.75 (s, 3H), 2.89 (br. s, 2H), 3.43 (m, 1H), 3.52 (m, 1H), 3.70 (m, 1H), 3.90 (1H,d), 4.40 (br. d, 1H), 5.49 (s, 2H), 5.54 (s, 1H), 7.02 (d, 2H), 7.44 (s, 1H), 7.49 (d, 2H), 7.53 (m, 1H), 7.71 (m, 1H), 7.91 (d, 1H), 8.08 (d, 1H).
iv) Methyl {3-tert-butoxycarbonylamino-3-[4-(2-methylquinolin-4-ylmethoxy)phenyl]-2-oxopyrrolidin-1-yl}acetate (525 mg, 1.01 mmol) was dissolved in anhydrous DCM (10 ml) and the solution cooled to −78° C. To this was added a solution of DIBAL (1.0M in DCM, 2.0 mmol, 2.0 ml) dropwise over 2 min. The solution was stirred at −78° C. for 2.5 h, before adding a further portion of DIBAL (1.0M in DCM, 1.0 mmol, 1.0 ml). The reaction mixture was stirred for a further 30 min before quenching with saturated ammonium chloride solution (15 ml) and allowing to warm to RT. The solution was then diluted with water (20 ml) and DCM (20 ml). This was then filtered and the organic phase dried (Na₂SO₄) and evaporated to give the crude aldehyde (370 mg) which was used without further purification; MS: 490.
v) To a stirred solution of tert-butyl [3-[4-(2-methylquinolin-4-ylmethoxy)phenyl]-2-oxo-1-(2-oxoethyl)pyrrolidin-3-yl]carbamate (365 mg, 0.75 mmol) in EtOH (5 ml) and water (5 ml) was added ammonium carbonate (430 mg, 4.5 mmol) and potassium cyanide (98 mg, 1.5 mmol). The mixture was heated to 65° C. for 2 h before addition of a second portion of ammonium carbonate (430 mg, 4.5 mmol). The reaction was heated for further 1 h. The reaction mixture was allowed to cool and then evaporated. The residue was partitioned between DCM (20 ml) and water (30 ml). The aqueous phase extracted with DCM (20 ml) and the combined organic phases dried (Na₂SO₄) and evaporated to a white foam. The crude product was purified by chromatography (Flashmaster II, 20 g silica bond elute, eluent 2% to 20%MeOH/DCM) to give the product, as a mixture of 2 diasteoisomers (186 mg, 0.33 mmol).

### Example 5

5-[3-(4-Benzyloxyphenyl)-3-methyl-2-oxopyrrolidin-1-ylmethyl]imidazolidine-2,4-dione

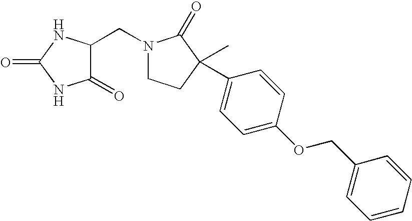

To a stirred solution of [3-(4-benzyloxyphenyl)-3-methyl-2-oxopyrrolidin-1-yl]acetaldehyde (343 mg, 1.06 mmol) in EtOH (5 ml) and water (5 ml) was added ammonium carbonate (610 mg, 6.35 mmol) and potassium cyanide (140 mg, 2.15 mmol). The mixture was heated to reflux for 3 h. The solution was allowed to cool and evaporated. The residue was partitioned between EtOAc (20 ml) and water (20 ml). The organic phase was washed with brine (20 ml), dried (Na₂SO₄) and evaporated. The crude product was purified by chromatography (Flashmaster II, 20 g silica bond elute, eluent 0%→10% MeOH in DCM) to give the product, as a 1:1 mixture of diasteoisomers, as a white foam (64 mg, 0.16 mmol); NMR 1.38 (s, 3H), 2.07 (m, 1H), 2.26 (m, 1H), 3.17-3.66 (m, 4H), 4.25 (s, 1H), 5.08 (s, 2H), 6.92-6.96 (m, 2H), 7.27-7.45 (m, 7H), 8.02 (s, 0.5H), 8.05 (s, 0.5H), 10.70 (s, 1H); MS: 394.

The starting material [3-(4-benzyloxyphenyl)-3-methyl-2-oxopyrrolidin-1-yl]acetaldehyde was prepared as follows:
i) Methyl [3-(4-benzyloxyphenyl)-3-methyl-2-oxopyrrolldin-1-yl]acetate (440 mg, 1.25 mmol) (example 2 step i)) was dissolved in DCM and cooled to −78° C. A solution of DIBAL (1.0M in DCM, 2.5 ml, 2.5 mmol) was added and the reaction mixture stirred at −78° C. for 1 h. The reaction was quenched by pouring onto sodium sulphate decahydrate. The resultant suspension was filtered and evaporated to give [3-(4-benzyloxyphenyl)-3-methyl-2-oxopyrrolidin-1-yl]acetaldehyde as an oil which was used in the next stage without further purification; MS: 324.

### Example 6

5-{3-Methyl-3-[4-(2-methylquinolin-4-ylmethoxy)phenyl]-2oxopyrrolidin-1-ylmethyl}-5-phenylimidazolidine-2,4-dione

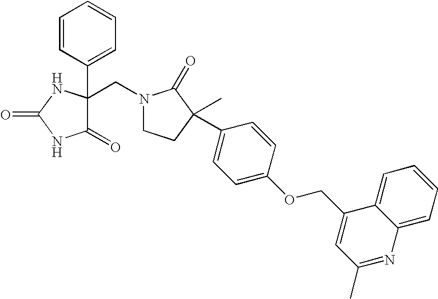

To a stirred solution of 3-methyl-3-[4-(2-methylquinolin-4-ylmethoxy)phenyl]-1-(2-oxo-2-phenylethyl)pyrrolidin-2-one (90 mg, 0.19 mmol) in EtOH (2 ml) and water (2 ml) was added ammonium carbonate (110 mg, 1.15 mmol) and potassium cyanide (25 mg, 0.38 mmol). The mixture was heated to 56° C. for 10 d. Silica gel (1 g) was added and the suspension evaporated. The resultant powder was applied to the top of a 5 g bond elute and chromatographed (Flashmaster II, eluent EtOAc) to give product of low purity (24 mg). This was further purified by preparative TLC to give the title compound (5 mg, 0.009 mmol) as a 1:1 mixture of diasteoisomers. MS: 535.

The starting material 3-methyl-3-[4-(2-methylquinolin-4-ylmethoxy)phenyl]-1-(2-oxo-2-phenylethyl)pyrrolidin-2-one was prepared as follows:
i) To a solution of methyl 2-(4-benzyloxyphenyl)-2-methyl-4-oxobutanoate (4.90 g, 15.7 mmol) in 1,2-dichloroethane (100 ml) was added 2,2-dimethyl-1,3-dioxolan-4-ylmethylanine (3.3 ml, 25.4 mmol). The resultant solution was stirred at RT for 60 min before addition of sodium triacetoxyborohydride (5.3 g, 25 mmol). The reaction mixture was stirred for a further 1 h and stood at RT overnight before addition of DCM (100 ml) and brine (100 ml). The organic phase was washed with saturated sodium bicarbonate solution (100 ml), dried (Na₂SO₄) and evaporated. The resultant oil (6.53 g) was dissolved in EtOH (100 ml) and placed under an argon atmosphere. Cyclohexene (16 ml, 160 mmol) and 10% palladium on charcoal (2.0 g) were added and the resultant mixture heated to reflux for 2.5 h. The reaction mixture was allowed to cool, filtered and evaporated to an oil (5.54 g). The crude product was dissolved in DMSO (60 ml). To this caesium carbonate (10.25 g, 31.5 mmol), tetra-n-butylammonium iodide (5.8 g, 15.7 mmol) and 4-chloromethyl-2-methylquinoline (3.0 g, 15.7 mmol) were added and the mixture heated to 60° C. for 40 min. The reaction mixture was partitioned between EtOAc (200 ml) and brine (100 ml). The organic phase was washed with brine (2×100 ml), dried and evaporated. The crude product was purified by chromatography (Flashmaster II, eluent 100% EtOAc) to give 1-(2,2-dimethyl-[1,3]-dioxolan-4-ylmethyl)-3-methyl-3-[4-(2-methylquinolin-4-ylmethoxy)phenyl]pyrrolidin-2-one as an oil (3.74 g, 8.1 mmol) as a 1:1 mixture of diastereoisomers; NMR 1.25 (s, 3H), 1.30 (s, 1.5H), 1.35 (s, 1.5H), 1.388 (s, 1.5H), 1.393 (s, 1.5H), 2.09 (m, 1H), 2.30 (m, 1H), 2.67 (s, 3H), 3.27-3.48 (m, 4H), 3.58 (m, 1H), 3.97 (m, 1H), 4.22 (m, 1H), 5.59 (s, 2H), 7.08 (d, 1H), 7.09 (d, 1H), 7.31-7.35 (m, 2H), 7.55 (m, 1H), 7.58 (m, 1H), 7.75 (m, 1H), 7.97 (d, 1H), 8.11 (d, 1H); MS: 461.
ii) 1-(2,2-Dimethyl-[1,3]-dioxolan-4-ylmethyl)-3-methyl-3-[4-(2-methylquinolin-4-ylmethoxy)phenyl]pyrrolidin-2-one was dissolved in hydrochloric acid (2M, 40 ml) and left to stand for 20 min, during which time a thick white precipitate formed. The suspension was basified with saturated sodium bicarbonate solution and extracted with DCM (2×150 ml). The organic phase was dried (Na₂SO₄) and evaporated to give 1-(2,3-dihydroxypropyl)-3-methyl-3-[4-(2-methylquinolin-4-ylmethoxy)phenyl]pyrrolidin-2-one (3.3 g, 7.81 mmol); NMR 1.39 (s, 3H), 2.08 (m, 1H), 2.30 (m, 1H), 2.67 (s, 3H), 3.10-3.44 (m, 6H), 3.66 (m, 1H), 4.52-4.57 (m, 1H), 4.76-4.78 (m, 1H), 5.58 (s, 2H), 7.078 (d, 1H), 7.084 (d, 1H), 7.33 (d, 1H), 7.34 (d, 1H), 7.56 (s, 1H), 7.59 (m, 1H), 7.75 (m, 1H), 7.97 (d, 1H), 8.10 (d, 1H); MS: 421.
iii) 1-(2,3-Dihydroxypropyl)-3-methyl-3-[4-(2-methylquinolin-4-ylmethoxy)phenyl]pyrrolidin-2-one (1.65 g, 3.93 mmol) was dissolved in MeOH (50 ml) and water (10 ml). Sodium periodate was added to the solution and the mixture left to stand for 30 min, during which time a thick white precipitate formed. MeOH was evaporated and the residue partitioned between saturated sodium bicarbonate (50 ml) and DCM (50 ml). The aqueous phase was extracted with DCM (2×50 ml). The combined organic phases were dried (Na₂SO₄) and evaporated. The resultant oil was redissolved in toluene (100 ml) and evaporated. This was repeated a further 5 times to give {3-methyl-3-[4-(2-methylquinolin-4-ylmethoxy)phenyl]-2-oxopyrrolidin-1-yl}acetaldehyde as an oil (1.52 g, 3.92 mmol). MS: 389.
iv) 3-Methyl-3-[4-(2-methylquinolin-4-ylmethoxy)phenyl]-2-oxopyrrolidin-1-yllacetaldehyde (210 mg, 0.54 mmol) was dissolved in THF (5 ml) in cooled to 0° C. To this solution was added a solution of phenyl magnesium bromide (1.0 M in THF, 0.65 ml) and solution stirred at 0° C. for 1 h. A further portion of phenyl magnesium bromide (1.0 M in THF, 0.33 ml) was added and the ice-bath removed. The solution was stirred at RT for 20 min before quenching with saturated ammonium chloride (10 ml) and portioning between EtOAc (50 ml) and brine (50 ml). The organic phase was dried (Na₂SO₄) and evaporated. The crude product was purified by chromatography (Flashmaster II, 10 g silica bond elute, eluent 70%→100% EtOAc in isohexane) to give 1-(2-hydroxy-2-phenylethyl)-3-methyl-3-[4-(2-methylquinolin-4-ylmethoxy)phenyl]pyrrolidin-2-one as a yellow oil (120 mg, 0.26 mmol); MS: 467.
v) 1-(2-Hydroxy-2-phenylethyl)-3-methyl-3-[4-(2-methylquinolin-4-ylmethoxy)phenyl]pyrrolidin-2-one (120 mg, 0.26 mmol) was dissolved in DCM (4 ml). NMO (53 mg, 0.39 mmol) and 4A molecular sieves (300 mg) were added. The reaction was stirred for 10 min before addition of TPAP (6 mg). The reaction was stirred for 30 min and poured onto a 5 g silica bond elute and eluted with EtOAc to give 3-methyl-3-[4-(2-methylquinolin-4-ylmethoxy)phenyl]-1-(2-oxo-2-phenylethyl)pyrrolidin-2-one as an oil (90 mg, 0.19 mmol); MS: 465.

### Example 7

5-Isobutyl-5-{3-methyl-3-[4-(2-methylquinolin-4-ylmethoxy)phenyl]-2-oxopyrrolidin-1-ylmethyl}imidazolidine-2,4-dione

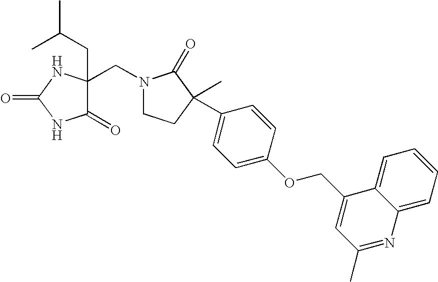

An analogous method to that described in Example 6 was used except that isobutyl magnesium chloride (2.0M in THF) was used instead of phenyl magnesium bromide (1.0M in THF) to give 5-isobutyl-5-{3-methyl-3-[4-(2-methylquinolin-4-ylmethoxy)phenyl]-2-oxopyrrolidin-1-ylmethyl}imidazolidine-2,4-dione (6 mg, 0.011 mmol); MS:515.

### Example 8

5-[(3-{4-[(2,5-dinethylbenzyl)oxy]phenyl}-3-methyl-2-oxopyrrolidin-1-yl)methyl]imidazolidine-2,4-dione

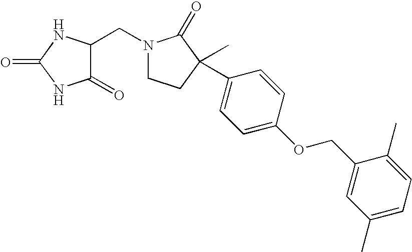

An analogous method to that described in Example 6 was used to give 5-[(3-{4-[(2,5-dimethylbenzyl)oxy]phenyl}-3-methyl-2-oxopyrrolidin-1-yl)methyl]imidazolidine-2,4-dione 68 mg (0.161 mmol); NMR (DMSOd6) 1.4 (m, 3H), 2.1 (m, 1H), 2.3 (m, 4H), 3.3 (m, 6H), 3.4-3.5 (m, 3H), 3.6 (m, 1H), 4.25 (t, 3H), 5.0 (s, 2H), 6.95 (m, 2H), 7.05-7.15 (m, 2H), 7.2 (s, 1H), 7.3 (m, 2H), 8.1 (d, 1H), 10.8 (s, 1H); MS 422.

The starting material was prepared from methyl 2-(4-benzyloxyphenyl)-2-methyl-4-oxobutanoate as highlighted in example 6 using steps i), ii) and iii), except that 4-chloromethyl-2-methylquinoline was replaced with 2,5-dimethylbenzyl chloride in step i).

### Example 9

5-[(3-{4-[(3,5-difluorobenzyl)oxy]phenyl}-3-methyl-2-oxopyrrolidin-1-yl)methyl]imidazolidine-2,4-dione

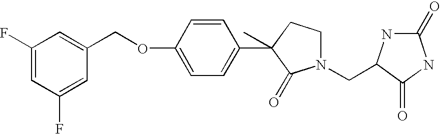

An analogous method to that described in Example 6 was used to give 5-[(3-{4-[(3,5-difluorobenzyl)oxy]phenyl}-3-methyl-2-oxopyrrolidin-1-yl)methyl]imidazolidine-2,4-dione 60 mg, 0.14 mmol; NMR (DMSOd6) 1.35 (d, 2H), 2.1 (m, 1H), 2.2 (m, 2H), 3.2-3.7 (m, 4H), 4.2 (m, 1H), 5.1 (s, 2H), 6.95 (m, 2H), 7.2 (m, 3H) 7.3 (s, 2H), 8.1 (d, 1H) 10.7 (s, 1H); MS 430.

The starting material was prepared from methyl 2-(4-benzyloxyphenyl)-2-methyl-4-oxobutanoate as highlighted in example 6 using steps i), ii) and iii), except that 4-chloromethyl-2-methylquinoline was replaced with 3,5-difluorobenzyl chloride in step i).

### Example 10

5-({3-[4-(but-2-yn-1-yloxy)phenyl]-3-methyl-2-oxopyrrolidin-1-yl}methyl)imidazoldine-2,4-dione

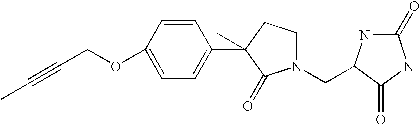

An analogous method to that described in Example 6 was used to give 5-({3-[4-(but-2-yn-1-yloxy)phenyl]-3-methyl-2-oxopyrrolidin-1-yl}methyl)imidazolidine-2,4-dione (52 mg, 0.15 mmol); NMR (DMSOd6) 1.4 (m, 3H), 1.8 (s, 3H), 2.1 (m, 1H), 2.3 (m, 1H), 3.2-3.7 (m, 4H), 4.25 (s, 1H), 4.7 (s, 2H), 6.9 (m, 2H), 7.3 (m, 2H), 8.0 (d, 1H), 10.7 (s, 1m); MS 365.

The starting material was prepared from methyl 2-(4-benzyloxyphenyl)-2-methyl-4-oxobutanoate as highlighted in Example 6 using steps i), ii) and iii), except that 4-chloromethyl-2-methylquinoline was replaced with 1-chlorobut-2-yne in step i).

### Example 11

5-Hydroxymethyl-5-{3-methyl-3-[4-(2-methylquinolin-4-ylmethoxy)pheny]-2-oxopyrrolidin-1-ylmethyl}inidazolidine-2,4-dione

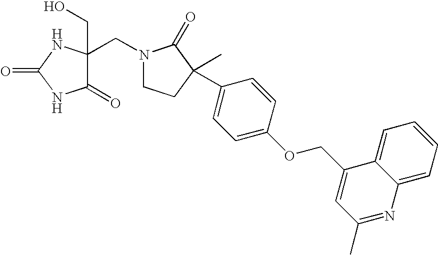

To a stirred solution 1-(3-hydroxy-2-oxopropyl)-3-methyl-3-[4-(2-methylquinolin-4-ylmethoxy)phenyllpyrrolidin-2-one (106 mg, 0.25 mmol) in EtOH (1 ml) and water (1 ml) was added ammonium carbonate (144 mg, 1.5 mmol) and potassium cyanide (32 mg, 0.49 mmol). The mixture was heated to 56° C. for 90 min. Silica gel (1 g) was added and the suspension evaporated. The resultant powder was applied to the top of a 5 g bond elute and chromatographed Flashmaster II, eluent 0-10% EtOH in DCM) to give product as a 1:1 mixture of diastereoisomers (60 mg, 0.12 mmol); MS: 489.

The starting material 1-(3-hydroxy-2-oxopropyl)-3-methyl-3-[4-(2-methylquinolin-4-ylmethoxy)phenyl]pyrrolidin-2-one was prepared as follows:
i) To a solution of 1-(2,3-dihydroxypropyl)-3-methyl-3-[4-(2-methylquinolin-4-ylmethoxy)phenyl]pyrrolidin-2-one (1.24 g, 2.95 mmol) (example 6 step ii)) in DCM (30 ml) was added imidazole (300 mg, 4.4 mmol) and tert-butyldimethylsilyl chloride (490 mg, 3.25 mmol). The resultant solution was stirred at RT for 3 h. The solvent was evaporated and the oily residue chromatographed (flashmaster II, 40-100% EtOAc in isohexane) to give 1-[3-(tert-butyldimethylsilyloxy)-2-hydroxypropyl]-3-methyl-3-[4-(2-methylquinolin-4-ylmethoxy)phenyl]pyrrolidin-2-one as a colourless oil (1.15 g, 2.15 mmol); MS: 535.
ii) To a solution of 1-[3-(tert-butyldimethylsilyloxy)-2-hydroxypropyl]-3-methyl-3-[4-(2-methylquinolin-4-ylmethoxy)phenyl]pyrrolidin-2-one (1.15 g, 2.15 mmol) in DCM (40 ml) was added NMO (435 mg, 3.22 mmol) and 4A molecular sieves (2.0 g). The suspension was stirred for 10 min at RT before addition of TPAP (40 mg). The reaction mixture was stirred for a further 30 min before pouring onto a 10 g silica gel bond elute and eluted with EtOAc (50 ml) to give 1-[3-(tert-butyldimethylsilyloxy)-2-oxopropyl]-3-methyl-3-[4-(2-methylquinoln-4-ylmethoxy)phenyl]pyrrolidin-2-one (980 mg, 1.8 mmol); NMR 0.00 (s, 6H), 0.83 (s, 9H), 1.36 (s, 3H), 2.07 (m, 1H), 2.25 (m, 1H), 2.60 (s, 3H), 3.26 (m, 2H), 4.17 (ABq, 2H), 4.28 (s, 2H), 5.52 (s, 2H), 7.02 (d, 2H), 7.29 (d, 2H), 7.49 (s, 1H), 7.51 (m, 1H), 7.67 (m, 1H), 7.90 (d, 1H), 8.03 (d, 1H); MS: 533.
iii) Acetyl chloride (2 ml) was added to MeOH (20 ml) at 0° C. then allowed to warm to RT. To this was added 1-[3-(tert-butyldimethylsilyloxy)-2-oxopropyl]-3-methyl-3-[4-(2-methylquinolin-4-ylmethoxy)phenyl]pyrrolidin-2-one (980 mg, 1.8 mmol). The reaction mixture was stirred at RT for 10 min and then evaporated to a cream solid. The solid was dissolved in saturated sodium bicarbonate (50 ml) and extracted with DCM (2×50 ml). The combined organic phases were dried and evaporated to give 1-(3-hydroxy-2-oxopropyl)-3-methyl-3-[4-(2-methylquinolin-4-ylmethoxy)phenyl]pyrrolidin-2-one as an oil (820 mg, 1.96 mmol); NMR 1.47 (s, 3H), 2.19 (m, 1H), 2.36 (m, 1H), 2.70 (s, 3H), 3.30 (m, 2H), 4.17 (d, 2H), 4.30 (ABq, 2H), 5.33 (t, 1H), 5.63 (s, 2H), 7.13 (d, 2H), 7.41 (d, 2H), 7.60 (s, 1H), 7.62 (m, 1H), 7.78 (m, 1H), 8.00 (d, 1H), 8.14 (d, 1H); MS: 419.

### Example 12

5-[(3-{4-[(2,5-dimethylbenzyl)oxy]phenyl}-3-methyl-2-oxopyrrolidin-1-yl)methyl]-5-methylimidazolidine-2,4-dione

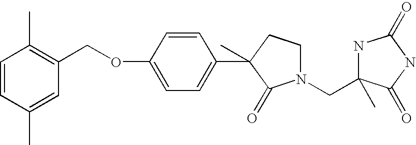

An analogous method to that described in Example 3 was used except that 4-chloromethyl-2-methylquinoline was replaced with 2,5-dimethylbenzyl chloride in step i) to afford 5-[(3-{4-[(2,5-dimethylbenzyl)oxy]phenyl}-3-methyl-2-oxopyrrolidin-1-yl)methyl]-5-methylimidazolidine-2,4-dione as a white solid; NMR (DMSO) 1.24 (d, 3H), 1.36 (d, 3H), 2.05 (m, 1H), 2.23 (m, 1H), 2.27 (s, 6H), 3.25 (m, 2H), 3.47 (q, 1H), 4.995 (d, 2H), 6.95 (t, 2H), 7.05 (dd, 1H), 7.10 (d, 1H), 7.22 (d, 1H), 7.265 (dd, 2H), 7.989 (d, 1H), 10.67 (d, 1H); MS: 436 (MH+).

### Example 13

5-({3-methyl-3-[4-(1-naphthylmethoxy)phenyl]-2-oxopyrrolidin-1-yl}methyl)inidazolidine-2,4-dione

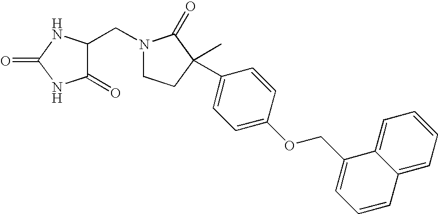

An analogous method to that described in Example 3 was used to give 5-({3-methyl-3-[4-(1-naphthylmethoxy)phenyl]-2-oxopyrrolidin-1-yl}methyl)imidazolidine-2,4-dione as a fawn solid (22 mg, 0.05 mmol); NMR DMSOd6 2.08 (m, 1H), 2.25 (m, 1H), 3.20-3.66 (m, 4H), 4.25 (d, 1H), 5.50 (s, 2H), 7.00 (d, 2H), 7.29 (d, 2H), 7.43-7.60 (m, 3H), 7.65 (d, 1H), 7.88-8.12 (m, 4H), 7.67 (d, 1H), 10.67 (s, 1H); MS 466 (MNa+).

The starting material was prepared from 2-(4-benzyloxy-phenyl)-2-methyl-4-oxo-butyric acid methyl ester as highlighted in example 6 using steps i), ii) and iii), except that 4-chloromethyl-2-quinoline was replaced with 1-(chloromethyl)naphthalene.

### Example 14

5-({3-amino-3-[4-(1-naphthylmethoxy)phenyl]-2-oxopyrrolidin-1-yl}methyl)inidazolidine-2,4-dione

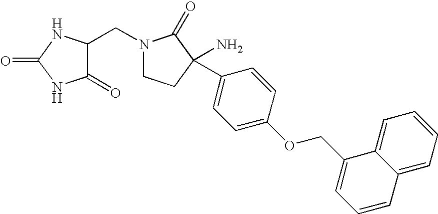

To a stirred solution of tert-butyl {1-[(2,5-dioxoimidazolidin-4-yl)methyl]-3-[4-(1-naphthylmethoxy)phenyl]-2-oxopyrrolidin-3-yl}carbamate (100 mg, 0.18 mmol) in DCM (5 ml) was added TFA (0.5 ml). The reaction was stirred for 90 min, evaporated to dryness and Durified by reverse phase HPLC on a Phenomenex C-18 prep column eluting with an acetonitrile:water:TFA gradient, which on further purification on a 10 g SCX isolute column gave the product (10 mg, 0.02 mmol) as a mixture of diasteroisomers; NMR DMSOd6 2.10-2.23 (m, 2H), 3.24-3.72 (m, 4H), 4.31 (t, 1H), 5.54 (d, 2H), 7.04 (t, 2H), 7.37 (d, 2H), 7.50-7.61 (m, 3H), 7.67 (d, 1H), 7.93-8.00 (m, 2H), 8.05-8.10 (m, 2H), 10.75 (bs, 1H); MS: 467 (MNa+).

The starting material tert-butyl {1-[(2,5-dioxoimidazolidin-4-yl)methyl]-3-[4-(1-naphthylmethoxy)phenyl]-2-oxopyrrolidin-3-yl}carbamate was prepared as follows:
i) To a solution of methyl 2-(4-benzyloxyphenyl)-2-tert-butoxycarbonylamino-4-oxo-butanoate (1.64 g, 3.97 mmol) (example 4) in 1,2-dichloroethane (23 ml) was added 2,2-dimethyl-1,3-dioxolan-4-methylamine (0.52 ml, 4.01 mmol). The resultant solution was stirred at RT for 60 min before addition of sodium triacetoxyborohydride (1.86 g, 8.78 mmol). The reaction mixture was stirred for a further 1 h and stood at RT for 2 days before addition of DCM (25 ml) and brine (25 ml). The organic phase was washed with saturated sodium bicarbonate solution (25 ml), dried (Na₂SO₄) and evaporated to give an oil. The product was purified by flash chromatography on silica gel (isohexane:ether, 50:50) to give tert-butyl {3-[4-(benzyloxy)phenyl]-1-[(2,2-dimethyl-1,3-dioxolan-4-yl)methyl]-2-oxopyrrolidin-3-yl}carbamate as a mixture of diastereoisomers (1.21 g, 2.44 mmol); NMR DMSOd6 1.24 (s, 6H), 1.33 (s, 9H), 2.77 (d, 2H), 3.33-3.64 (m, 6H), 3.92 (m, 1H), 4.14 (m, 1H), 4.98 (s, 2H), 5.46 (s, 1H), 6.86 (d, 2H), 7.22-7.37 (m, 7H).
ii) A solution of tert-butyl {3-[4-(benzyloxy)phenyl]-1-[(2,2-dimethyl-1,3-dioxolan4-yl)methyl]-2-oxopyrrolidin-3-yl}carbamate (1.20 g, 2.42 mmol) in (THF:2N HCl, 50 ml) was stirred at RT for 2 d, evaporated to near dryness and treated with water (25 ml) and saturated aqueous sodium carbonate added to pH8. The reaction mixture was extracted with DCM, dried (MgSO₄) and evaporated. The crude was purified by flash chromatography (20 g isolute silica column, eluent 0%→10% MeOH in DCM) to give 3-amino-3-[4-(benzyloxy)phenyl]-1-(2,3-dihydroxypropyl)pyrrolidin-2-one as a mixture of diastereoisomers (0.4 g, 1.12 mmol); MS: 340 (MNH3+).
iii) To a stirred and cooled (ice/water) mixture of 3-amino-3-[4-(benzyloxy)phenyl]-1-(2,3-dihydroxypropyl)pyrrolidin-2-one (0.4 g, 1.12 mmol), THF (5 ml), water (5 ml) and di-tert-butyl dicarbonate (0.27 g, 1.24 mmol) was added potassium carbonate (0.3 g, 2.17 mmol) portionwise. The reaction mixture was stirred at RT overnight, evaporated, extracted with DCM, dried (MgSO₄) and evaporated to dryness to give tert-butyl [3-[4-(benzyloxy)phenyl]-1-(2,3-dihydroxypropyl)-2-oxopyrrolidin-3-yl]carbamate as a mixture of diastereoisomers (0.57 g, 1.25 mmol) which was used directly in the next step.
iv) A mixture of tert-butyl [3-[4-(benzyloxy)phenyl]-1-(2,3-dihydroxypropyl)-2-oxopyrrolidin-3-yl]carbamate (0.57 g, 1.25 mmol), cyclohexene (1.27 ml, 12.5 mmol), EtOH (10 ml) and 10% palladium on charcoal was stirred and refluxed for 2 h and then left for 18 h at RT. The reaction mixture was filtered through celite, loaded onto a 20 g flash silica isolute column, eluted with DCM, ether, EtOAc and 1/9 MeOH/DCM to give tert-butyl (1-(2,3-dihydroxypropyl)-3-(4-hydroxyphenyi)-2-oxopyrrolidin-3-yl]carbamate as a mixture of diastereoisomers (300 mg, 0.82 mmol); NMR CDCl₃ 1.41 (s, 91), 2.70 (m, 1H), 2.89 (m, 1H), 3.3-3.6 (m, 6H), 3.8-3.98 (m, 1H), 5.43 (d, 1H), 6.72 (d, 2H), 7.27 (d, 2H); MS: 389 (MNa+).
v) A mixture of tert-butyl [1-(2,3-dihydroxypropyl)-3-(4-hydroxyphenyl)-2-oxopyrrolidin-3-yl]carbamate (150 mg, 0.41 mmol), DMSO (2 ml), caesium carbonate (0.266 g, 0.82 mmol), tetrabutyl ammonium iodide (0.151 g, 0.409 mmol) and 1-chloromethylnapthalene (61 μl, 0.407 mmol) was stirred and heated at 60° C. for 90 min. After cooling, EtOAc (25 ml) was added and the reaction mixture washed with brine, dried (MgSO₄) and evaporated. The crude product was purified by chromatography (10 silica isolute column, eluant 0%→7% MeOH/DCM) to give tert-butyl {1-(2,3-dihydroxypropyl)-3-[4-(1-naphthylmethoxy)phenyl]-2-oxopyrrolidin-3-yl}carbamate as a mixture of diastereoisomers (0.14 g, 0.28 mmol); MS: 529 (MNa+).
vi) To a solution of tert-butyl {1-(2,3-dihydroxypropyl)-3-[4-(1-naphthylmethoxy)phenyl]-2-oxopyrrolidin-3-yl}carbamate (140 mg, 0.28 mmol) in DCM (1.0 ml), MeOH (3.5 ml) and water (0.7 ml) was added sodium periodate (59 mg, 0.276 mmol). The reaction mixture was stirred for 90 min, evaporated, water (10 ml) and EtOAc (10 ml) added and stirred for a further 30 min. The organic layer was dried (MgSO₄) and evaporated to yield tert-butyl [3-[4-(1-naphthylmethoxy)phenyl]-2-oxo-1-(2-oxoethyl)pyrrolidin-3-yl]carbamate (90 mg, 0.19 mmol); MS: 529 (M/Hemi acetal/Na+).
vii) To a solution of tert-butyl [3-[4-(1-naphthylmethoxy)phenyl]-2-oxo-1-(2-oxoethyl)pyrrolidin-3-yl]carbamate (110 mg. 0.316 mmol) in EtOH (2.5 ml) and water (2.5 ml) was added ammonium carbonate (182 mg, 1.89 mmol) and potassium cyanide (41 mg, 0.63 mmol). The reaction mixture was stirred and heated at 60° C. for 2 h, left for 2 d at RT, then evaporated to dryness. The resultant residue was dissolved in DCM, filtered and evaporated to give the product as a gum (100 mg, 0.84 mmol); MS: 576 (MNa+), 543 (M−).

1. A compound of formula (1) or a pharmaceutically acceptable salt thereof wherein: 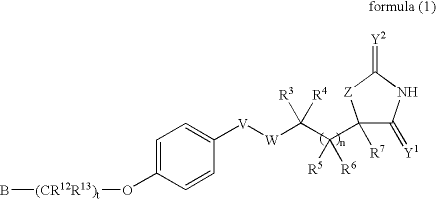
Y¹ and y² are both O;
z is NR⁸, O or S;
n is 0 or 1;
W is CR¹R² or a bond;
V is a group of formula (A): 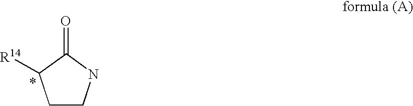
where the group of formula (A) is bonded through nitrogen to W of formula (1) and through carbon * to phenyl of formula (1);
t is 0 or 1;
B is a group selected from aryl, heteroaryl and heterocyclyl where each group is optionally substituted by one or more groups independently selected from nitro, trifluoromethyl, trifluoromethoxy, halo, cyano, C₁₋₄alkyl optionally substituted by R⁹ or C₁₋₄alkoxy or one or more halo, C₂₋₄alkenyl optionally substituted by halo or R⁹, C₂₋₄alkynyl optionally substituted by halo or R⁹, C₃₋₆cycloalkyl optionally substituted by R⁹ or one or more halo), C₅₋₆cycloalkenyl optionally substituted by halo or R⁹, aryl optionally substituted by halo or C₁₋₄alkyl, heteroaryl optionally substituted by halo or C₁₋₄alkyl, heterocyclyl optionally substituted by C₁₋₄alkyl, —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl, heterocyclyl whereby this group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl and C₁₋₄alkoxy;
R¹ and R² are independently hydrogen or a group selected from C₁₋₆alkyl, C₂₋₆alkenyl, C₂₋₆alkynyl, C₃₋₆cycloalkyl and C₅₋₆cycloalkenyl which group may be optionally substituted by halo, cyano, hydroxy or C₁₋₄alkoxy;
R³, R⁴, R⁵ and R⁶ are independently hydrogen or a group selected from C₁₋₆alkyl, C₂₋₆alkenyl, C₂₋₆alkynyl, C₃₋₆cycloalkyl, C₅₋₆cycloalkenyl, aryl, heteroaryl and heterocyclyl which group is optionally substituted by one or more substituents independently selected from halo, nitro, cyano, trifluoromethyl, trifluoromethoxy, C₁₋₄alkyl, C₂₋₄alkenyl, C₂₋₄alkynyl, C₃₋₆cycloalkyl optionally substituted by one or more R¹⁷, aryl optionally substituted by one or more R¹⁷, heteroaryl optionally substituted by one or more R¹⁷, heterocyclyl, —OR¹⁸, —SR¹⁹, —SOR¹⁹, —SO₂R¹⁹, —COR¹⁹, —CO₂R¹⁸, —CONR¹⁸R²⁰, —NR¹⁶COR¹⁸, —SO₂NR¹⁸R²⁰ and —NR¹⁶SO₂R¹⁹;
or R¹ and R³ together with the carbon atoms to which they are attached form a saturated 3- to 7-membered ring optionally containing 1 or 2 heteroatoms groups selected from NH, O, S, SO and SO₂ where the ring is optionally substituted on carbon by C₁₋₄alkyl, fluoro or C₁₋₃alkoxy and/or nitrogen by C₁₋₄alkyl, —COC₁₋₃alkyl or —SO₂C₁₋₃alkyl;
or R³ and R⁴ together with the carbon atom to which they are attached form a saturated 3- to 7-membered ring optionally containing a heteroatom group selected from NH, O, S, SO and SO₂ where the ring is optionally substituted on carbon by C₁₋₄alkyl, fluoro or C₁₋₃alkoxy and/or nitrogen by C₁₋₄alkyl, —COC₁₋₃alkyl or —SO₂C₁₋₃alkyl;
or R³ and R⁵ together with the carbon atoms to which they are attached form a saturated 3- to 7-membered ring optionally containing a heteroatom group selected from NH, O, S, SO and SO₂ where the ring is optionally substituted on carbon by C₁₋₄alkyl, fluoro or C₁₋₃alkoxy and/or nitrogen by C₁₋₄alkyl, —COC₁₋₃alkyl or —SO₂C₁₋₃alkyl;
or R⁵ and R⁶ together with the carbon atom to which they are attached form a saturated 3- to 7-membered ring optionally containing a heteroatom group selected from NH, O, S, SO and SO₂ where the ring is optionally substituted on carbon by C₁₋₄alkyl, fluoro or C₁₋₃alkoxy and/or nitrogen by C₁₋₄alkyl, —COC₁₋₃alkyl or —SO₂C₁₋₃alkyl;
R⁷ is hydrogen or a group selected from C₁₋₆alkyl, C₂₋₆alkenyl, C₂₋₆alkynyl, heteroalkyl, C₃₋₇cycloalkyl, aryl, heteroaryl or heterocyclyl which group is optionally substituted by halo, C₁₋₄alkyl, C₁₋₄alkoxy, C₃₋₇cycloalkyl, heterocyclyl, aryl, heteroaryl and heteroalkyl; and wherein the group from which R⁷ may be selected is optionally substituted on the group and/or on its optional substituent by one or more substituents independently selected from halo, cyano, C₁₋₄alkyl, nitro, haloC₁₋₄alkyl, heteroalkyl, aryl, heteroaryl, hydroxyC₁₋₄alkyl, C₃₋₇cycloalkyl, heterocyclyl, C₁₋₄alkoxyC₁₋₄alkyl, haloC₁₋₄alkoxyC₁₋₄alkyl, —COC₁₋₄alkyl, —OR²¹, —CO₂R²¹, —SR²⁵, —SOR²⁵, —SO₂R²⁵, —NR²¹COR²², —CONR²¹R²² and —NHCONR²¹R²²;
or R³ and R⁷ together with the carbon atoms to which they are each attached and (CR⁵R⁶)ₙ form a saturated 5- to 7-membered ring optionally containing a heteroatom group selected from NH, O, S, SO and SO₂ where the ring is optionally substituted on carbon by C₁₋₄alkyl, fluoro or C₁₋₃alkoxy and/or nitrogen by C₁₋₄alkyl, —COC₁₋₃alkyl or —SO₂C₁₋₃alkyl;
R⁸ is hydrogen or methyl;
R⁹ and R¹⁰ are independently hydrogen, C₁₋₆alkyl or C₃₋₆cycloalkyl;
or R⁹ and R¹⁰ together with the nitrogen to which they are attached form a heterocyclic 4 to 7-membered ring;
R¹¹ is C₁₋₆alkyl or C₃₋₆cycloalkyl;
R¹² and R¹³ are independently selected from hydrogen, C₁₋₆alkyl and C₃₋₆cycloalkyl;
R¹⁴ is hydrogen, nitrile, —NR²³R²⁴ or C₁₋₄alkyl optionally substituted by halo, —OR²³ and —NR²³R²⁴;
R¹⁶, R²³ and R²⁴ are independently hydrogen or C₁₋₆alkyl;
R¹⁷ is selected from halo, C₁₋₆alkyl, C₃₋₆cycloalkyl and C₁₋₆alkoxy;
R¹⁸ is hydrogen or a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl which group is optionally substituted by one or more halo;
R¹⁹ and R²⁵ are independently a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl which group is optionally substituted by one or more halo;
R²⁰ is hydrogen, C₁₋₆alkyl or C₃₋₆cycloalkyl;
or R¹⁸ and R²⁰ together with the nitrogen to which they are attached form a heterocyclic 4- to 7-membered ring;
R²¹ and R²² are independently hydrogen, C₁₋₄alkyl, haloC₁₋₄alkyl, aryl and arylC₁₋₄alkyl.

2. A compound according to claim 1 wherein B is a group selected from aryl, heteroaryl and heterocyclyl where each group is optionally substituted by one or more groups independently selected from nitro, trifluoromethyl, trifluoromethoxy, halo, C₁₋₄alkyl optionally substituted by one or more halo, C₂₋₄alkynyl, heteroaryl, —OR⁹, cyano, —NR⁹R¹⁰, —CONR⁹R¹⁰ and —NR⁹COR¹⁰; or B is C₂₋₄alkenyl or C₂₋₄alkynyl optionally substituted by C₁₋₄alkyl, C₃₋₆cycloalkyl or heterocyclyl.

3. A compound according to claim 1 wherein B is phenyl, naphthyl, pyridyl, quinolinyl, isoquinolinyl, thienopyridyl, 1,8-naphthyridinyl, 2,3-methylenedioxyphenyl, 3,4-methylenedioxyphenyl, 1,6-naphthyridinyl, thienopyrimidinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl or isoindolinyl, where each is optionally substituted by one or more groups independently selected from nitro, trifluoromethyl, trifluoromethoxy, halo, C₁₋₄alkyl optionally substituted by one or more fluoro, C₂₋₄alkynyl, heteroaryl, —OR⁹, cyano, —NR⁹R¹⁰, —CONR⁹R¹⁰ and —NR⁹COR¹⁰; or B is vinyl or ethynyl optionally substituted by C₁₋₄alkyl.

4. A compound according to claim 2 wherein B is aryl, heteroaryl or C₂₋₄alkynyl optionally substituted by halo or C₁₋₄alkyl.

5. A compound according to claim 4 wherein B is 2-methylquinolin-4-yl or 2,5-dimethylphenyl.

6. A compound according to claim 1 wherein t is 1.

7. A compound according to claim 1 wherein R⁷ is selected from hydrogen, C₁₋₄alkyl, haloC₁₋₄alkyl, hydroxyC₁₋₄alkyl, C₁₋₄alkoxyC₁₋₄alkyl and aryl.

8. A compound according to claim 1 wherein R¹⁴ is hydrogen, methyl or amino.

9. A pharmaceutical composition comprising a compound according to claim 1 and a pharmaceutically-acceptable diluent or carrier.

10-11. (canceled)

12. A method of treating autoimmune disease, allergic/atopic diseases, transplant rejection, graft versus host disease, cardiovascular disease, reperfusion injury and malignancy which comprises administering a compound according to claim 1.

13. A process for preparing a compound according to claim 1, comprising the steps of converting a ketone or aldehyde of formula (2) into a compound of formula (1); 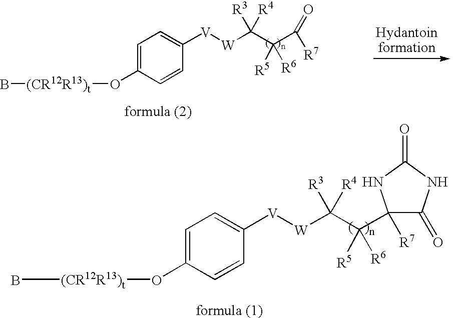
and thereafter if necessary:
i) converting a compound of formula (1) into another compound of formula (1);
ii) removing any protecting groups;
iii) forming a pharmaceutically acceptable salt or in vivo hydrolysable ester.
